(19) United States
(12) Patent Application Publication
(10) Pub. No.: US 2025/0170531 A1
(43) Pub. Date: May 29, 2025

(54) POLYAMIDE THIN FILM COMPOSITE MEMBRANES FOR NANOFILTRATION

(71) Applicant: KING FAHD UNIVERSITY OF PETROLEUM AND MINERALS, Dhahran (SA)

(72) Inventors: Abdul WAHEED, Dhahran (SA); Umair BAIG, Dhahran (SA); Isam H. ALJUNDI, Dhahran (SA)

(73) Assignee: KING FAHD UNIVERSITY OF PETROLEUM AND MINERALS, Dhahran (SA)

(21) Appl. No.: 18/521,628

(22) Filed: Nov. 28, 2023

(51) Int. Cl. B01D 69/12; B01D 61/02; B01D 69/02; B01D 69/10; B01D 71/42; B01D 71/56; C07C 29/76

(52) U.S. Cl. CPC B01D 69/1214; B01D 61/027; B01D 69/02; B01D 69/1071; B01D 71/421; B01D 71/56; C07C 29/76; B01D 2325/04; B01D 2325/06; B01D 2325/26

(57) ABSTRACT

A filtration membrane, including (in the following order) a thermoplastic substrate, a first layer including a polyacrylonitrile grafted with a diamine, and a second layer comprising reacted units of a tetramine and reacted units of a phthaloyl chloride. The tetramine has two secondary amine groups and two primary amine groups. The primary amine groups are separated from the secondary amine groups by a C2 or C3 aliphatic group. The reacted units of the tetramine and the reacted units of phthaloyl chloride form a polyamide. One or more amine groups of the polyacrylonitrile of the first layer are cross-linked to the second layer through one or more phthaloyl groups of the reacted units of the phthaloyl chloride. A method of nanofiltration using the filtration membrane.

### STATEMENT REGARDING PRIOR DISCLOSURE BY THE INVENTORS

Aspects of the present disclosure are described in Waheed, A., Baig, U., and Aljundi, I. H., “Fabrications of polyamide thin film composite membranes using aliphatic tetra-amines and terephthaloyl chloride crosslinker for organic solvent nanofiltration” published in Volume 13, Scientific Reports, which is incorporated herein by reference in its entirety.

### STATEMENT OF ACKNOWLEDGEMENT

Support provided by the Interdisciplinary Research Center for Membranes and Water Security, King Fahd University of Petroleum and Minerals, Saudi Arabia, through Project INMW2306 is gratefully acknowledged.

### BACKGROUND

### Technical Field

The present disclosure is directed towards a filtration membrane, more particularly, a polyamide thin film composite membrane with aliphatic tetramines and terephthaloyl chloride crosslinker and a nanofiltration method thereof.

### Description of Related Art

The “background” description provided herein is for the purpose of generally presenting the context of this disclosure. Work of the presently named inventors, to the extent it is described in this background section, as well as aspects of the description which may not otherwise qualify as prior art at the time of filing, are neither expressly nor impliedly admitted as prior art against the present disclosure.

Organic solvents are used in several industrial processes, and the use of membranes for recovering the solvents has evolved into an industrially viable process. Organic solvents are precious liquids which may be directly correlated to an industry's profits. Once used, organic solvents may dissolve foreign particles and become impure. Reusing these organic solvents is of economic interest, as it can lower the demand for fresh organic solvents. Further, industries may benefit from reusing organic solvents as it may reduce an industry's carbon footprint and overall adverse environmental impact. Hence, many industries are looking to constantly improve their processes to meet stringent environmental regulations and increase profit. Among the plurality of industries looking to improve their processes, the pharmaceutical industry consumes a large amount of organic solvents, where organic solvents account for 80 to 90% of the total mass in the processes involved in manufacturing pharmaceuticals. Furthermore, the most common solvent waste generated by pharmaceutical companies include, but may not be limited to, methanol, dichloromethane, toluene, acetonitrile, and chloroform. Among the different organic solvent wastes, methanol waste has been estimated to reach 44.8×10⁶ kg per year. Compared to conventional processes, such as distillation and adsorption, membrane-based separations are less energy intensive, more eco-friendly, have a lesser carbon footprint, and are more cost-effective for product isolation and concentration.

With the development of thin film composite (TFC) membranes, several separation processes have been optimized, which may be due to certain salient features of the TFC membranes, such as chemical and physical stability, adaptability to different feed compositions, and easy availability with a wide range of pore sizes for a desired application. TFC membranes consist of a thin selective layer deposited on a porous polymeric ultrafiltration (UF) support. Generally, the porous polymeric ultrafiltration support is prepared through wet phase inversion by using water as a non-solvent in the process. The active layer is then grown on the ultrafiltration support through interfacial polymerization (IP). Given the versatility of the IP process, a large variety of TFC membranes have been fabricated by either altering the chemistry of reacting monomers or changing other parameters of IP. The variations in the chemistry of the active layers of TFC membranes have led to many applications. A solvent resistant polyamide (PA) TFC membrane was prepared on a polyimide (PI) substrate and was made by using conventional meta-phenylenediamine (MPD) as an aqueous monomer and trimesoyl chloride (TMC) as a non-aqueous monomer during IP [Solomon, M. F. J., Bhole, Y. & Livingston, A. G. High flux membranes for organic solvent nanofiltration (OSN)-Interfacial polymerization with solvent activation. J. Memb. Sci. 423-424, 371-382 (2012)].

Another PA TFC membrane was reported using a green synthesis method by using polyethyleneimine (PEI) as an amine solution cross-linked with TMC on porous PAN support. The IP reaction was carried out using decanoic acid as the organic phase. The membrane was able to reject most of the tested dyes with a molecular weight cut-off (MWCO) of 650 g mol⁻¹ [Ong, C. et al. Green synthesis of thin-film composite membranes for organic solvent nanofiltration. ACS Sustain. Chem. Eng. 8(31), 11541-11548].

Recently, molecule-based design strategies have been explored for manipulating the pore structure and physicochemical properties of organic solvent nanofiltration/solvent-resistant nanofiltration (OSN/SRNF) membranes. A fluorine-containing diamine, 5-trifluoromethyl-1,3-phynelenediamine (TFMPD), was crosslinked with TMC and 4,4′-(hexafluoroisopropylidene)bis(benzoyl chloride) (HFBC) during IP for nonpolar organic solvent filtration [Alduraiei, F., Manchanda, P., Pulido, B., Szekely, G. & Nunes, S. P. Fluorinated thin-film composite membranes for nonpolar organic solvent nanofiltration. Sep. Purif. Technol. 279, 119777].

Since the presence of fluorine atoms in the active layer gives a hydrophobic feature to the membrane, the resulting membranes were used for cleaning non-polar solvents. The TFMPD-HFBC membrane showed a toluene permeance of 10 L m⁻² h⁻¹ bar⁻¹. Several amine monomers with 2.5-4 amines per molecule were crosslinked with binaphthalene-based di(acid chloride) through IP on a Matrimid support to tune the chemistry of the active layer of the TFC membrane by growing an active layer consisting of polymers of intrinsic microporosity (PIMs) [Thijs, M., Van Goethem, C., Vankelecom, I. F. J. & Koeckelberghs, G. Binaphthalene-based polymer membranes with enhanced performance for solvent-resistant nanofiltration. J. Memb. Sci. 606, 118066]. The fabricated membrane showed an increase in acetonitrile permeance by a factor of 20 compared to a conventional MPD/TMC membrane. By comparing the structure and performance of the different fabricated membranes, it was shown that changing the number of amine groups per molecule and altering the length of alkyl chains on binaphthalene-based di(acid chloride) altered the performance of the membranes.

In one such instance, Janus pathways were developed by using cyclodextrin (CD) in the membrane. The presence of CD in the constructed OSN/SRNF membrane allowed passage of both polar and non-polar solvents, which is attributed to the inner hydrophobic cavities and outer hydrophilic spaces of CD; however, the larger pore size of CD lowers the rejection of the membrane [Liu, J., Hua, D., Zhang, Y., Japip, S. & Chung, T. S. Precise molecular sieving architectures with Janus pathways for both polar and nonpolar molecules. Adv. Mater. 30(11), 1705933]. Similarly, a Janus membrane was developed by using β-cyclodextrin (BCD) as a monomer during IP on a polyelectrolytes-based interlayer developed through layer-by-layer self-assembly. The membrane was able to permeate both polar and non-polar solvents, with permeances reaching 5.8 LMH/bar for methanol and 7.0 LMH/bar for hexane, along with a 91.9% rejection of methyl orange [Li, X., Li, C., Goh, K., Chong, T. H. & Wang, R. Layer-by-layer aided β-cyclodextrin nanofilm for precise organic solvent nanofiltration. J. Memb. Sci. 652, 120466].

Although membranes have been developed in the past, there exists a need to develop new membranes with structural, physical, and chemical stability by tuning the chemistry of reacting monomers. Accordingly, an objective of the present disclosure is to describe a filtration membrane for organic solvents and a nanofiltration method thereof.

### SUMMARY

In an exemplary embodiment, a filtration membrane is disclosed. The filtration membrane includes (in the following order) a thermoplastic substrate, a first layer including a polyacrylonitrile grafted with a diamine, and a second layer comprising reacted units of a tetramine and reacted units of a phthaloyl chloride. Further, the tetramine has two secondary amine groups and two primary amine groups. Furthermore, the primary amine groups are separated from the secondary amine groups by a C2 or C3 aliphatic group; the reacted units of the tetramine and the reacted units of phthaloyl chloride form a polyamide. Moreover, one or more amine groups of the polyacrylonitrile of the first layer are cross-linked to the second layer through one or more phthaloyl groups of the reacted units of the phthaloyl chloride.

In some embodiments, the diamine is a hydrazine.

In some embodiments, the reacted units of tetramine and the reacted units of phthaloyl chloride bond through both the primary and secondary amino groups of the tetramine.

In some embodiments, the second layer is the form of nanoparticles with a diameter of 20 nm to 300 nm.

In some embodiments, the thermoplastic substrate is in the form of interwoven fibers with a diameter of 2 μm to 20 μm.

In some embodiments, the thermoplastic substrate has a thickness of 50 μm to 200 μm.

In some embodiments, the first layer includes crosslinked polyacrylonitrile in the form of vertical hollow tubes having a diameter of 1 μm to 50 μm and a length of 2 μm to 100 μm.

In some embodiments, the second layer has a thickness of 900 nm to 1000 nm.

In some embodiments, the tetramine is N,N′-bis(2-aminoethyl)-1,3-propanediamine (4A-3P).

In some embodiments, the tetramine is N,N′-bis(3-aminopropyl)ethylenediamine (4A).

In some embodiments, the membrane has a water contact angle (WCA) of 55° to 70°.

In some embodiments, the membrane has an average roughness of 10 nm to 30 nm.

In some embodiments, the membrane has a surface charge of −0.75 millivolts (mV) to −5.50 mV.

In another exemplary embodiment, a method for nanofiltration is disclosed. The method includes contacting the nanofiltration membrane with a solution. Further, the solution comprises one or more polar solvents, one or more non-polar solvents, and one or more solutes responsible for collecting a permeate passing through the filtration membrane to obtain a purified composition having a reduced amount of the solutes.

In some embodiments, the membrane has a permeate flux of 1 L m⁻² h⁻¹ to 80 L m⁻² h⁻¹ at a pressure of 4 bar.

In some embodiments, the polar solvent is methanol, and the membrane has a permeate flux of 7 L m⁻² h⁻¹ to 13 L m⁻² h⁻¹ at a pressure of 4 bar.

In some embodiments, the polar solvent is methanol, and the membrane has a rejection percentage of the pollutants of 4% to 100% based on an initial weight of the pollutants.

In some embodiments, the pollutants are selected from a group consisting of methylene blue (MB), eriochrome black T (EBT), and Congo red (CR).

In some embodiments, the pollutant is EBT, and the membrane has a rejection percentage of the EBT of 93% to 100% based on an initial weight of the EBT.

In some embodiments, the pollutant is CR, and the membrane has a rejection percentage of the CR of 98% to 100% based on an initial weight of the CR.

These are other aspects of non-limiting embodiments of the present disclosure will become apparent to those skilled in the art upon review of the following description of specific non-limiting embodiments of the disclosure in conjunction with the accompanying drawings. The foregoing general description of the illustrative present disclosure and the following detailed description thereof are merely exemplary aspects of the teachings of this disclosure and are not restrictive.

### BRIEF DESCRIPTION OF THE DRAWINGS

A more complete appreciation of this disclosure (including alternatives and/or variations thereof) and many of the attendant advantages thereof will be readily obtained as the same becomes better understood by reference to the following detailed description of the embodiments when considered in connection with the accompanying drawings, wherein:

FIG. 1A is a flowchart depicting a method for nanofiltration, according to certain embodiments;

FIG. 1B is a schematic illustration depicting different stages of fabrication of crosslinked polyacrylonitrile (PAN) support, 4A-terephthaloyl (TPC) @crosslinked PAN membrane, and 4A-3P-TPC@crosslinked PAN membrane, according to certain embodiments;

FIG. 2 shows attenuated total reflection-Fourier transform infrared (ATR-FTIR) spectra of crosslinked PAN support, 4A-TPC@crosslinked PAN membrane, and 4A-3P-TPC@crosslinked PAN membrane, according to certain embodiments;

FIG. 3A shows scanning electron microscopy (SEM) micrograph of the crosslinked PAN support at a scale of 5 μm, according to certain embodiments;

FIG. 3B shows SEM micrograph of the crosslinked PAN support at a scale of 1 μm, according to certain embodiments;

FIG. 3C shows SEM micrograph of the crosslinked PAN support at a scale of 500 nm, according to certain embodiments;

FIG. 3D shows SEM micrograph of the 4A-TPC@crosslinked PAN membrane at a scale of 5 μm, according to certain embodiments;

FIG. 3E shows SEM micrograph of the 4A-TPC@crosslinked PAN membrane at a scale of 1 μm, according to certain embodiments;

FIG. 3F shows SEM micrograph of the 4A-TPC@crosslinked PAN membrane at a scale of 500 nm, according to certain embodiments;

FIG. 3G shows SEM micrograph of the 4A-3P-TPC@crosslinked PAN membrane at a scale of 5 μm, according to certain embodiments;

FIG. 3H shows SEM micrograph of the 4A-3P-TPC@crosslinked PAN membrane at a scale of 1 μm, according to certain embodiments;

FIG. 3I shows SEM micrograph of the 4A-3P-TPC@crosslinked PAN membrane at a scale of 500 nm, according to certain embodiments;

FIG. 4A shows SEM micrograph of the bottom surface of the crosslinked PAN support at a scale of 5 μm, according to certain embodiments;

FIG. 4B shows SEM micrograph of the bottom surface of the crosslinked PAN support at a scale of 1 μm, according to certain embodiments;

FIG. 4C shows SEM micrograph of the bottom surface of the crosslinked PAN support at a scale of 500 nm, according to certain embodiments;

FIG. 4D is a SEM micrograph of the bottom surface of the 4A-TPC@crosslinked PAN membrane at a scale of 5 μm, according to certain embodiments;

FIG. 4E is a SEM micrograph of the bottom surface of the 4A-TPC@crosslinked PAN membrane at a scale of 1 μm, according to certain embodiments;

FIG. 4F shows SEM micrograph of the bottom surface of the 4A-TPC@crosslinked PAN membrane at a scale of 500 nm, according to certain embodiments;

FIG. 4G shows SEM micrograph of the bottom surface of the 4A-3P-TPC@crosslinked PAN membrane at a scale of 5 μm, according to certain embodiments;

FIG. 4H shows SEM micrograph of the bottom surface of the 4A-3P-TPC@crosslinked PAN membrane at a scale of 1 μm, according to certain embodiments;

FIG. 4I shows SEM micrograph of the bottom surface of the 4A-3P-TPC@crosslinked PAN membrane at a scale of 500 nm, according to certain embodiments;

FIG. 5A shows a cross-sectional SEM micrograph of the 4A-TPC@crosslinked PAN membrane at a scale of 100 μm, according to certain embodiments;

FIG. 5B shows a cross-sectional SEM micrograph of the 4A-TPC@crosslinked PAN membrane at a scale of 50 μm, according to certain embodiments;

FIG. 5C shows a cross-sectional SEM micrograph of the 4A-TPC@crosslinked PAN membrane at a scale of 10 μm, according to certain embodiments;

FIG. 5D shows a cross-sectional SEM micrograph of the 4A-3P-TPC@crosslinked PAN membrane at a scale of 100 μm, according to certain embodiments;

FIG. 5E shows a cross-sectional SEM micrograph of the 4A-3P-TPC@crosslinked PAN membrane at a scale of 50 μm, according to certain embodiments;

FIG. 5F shows a cross-sectional SEM micrograph of the 4A-3P-TPC@crosslinked PAN membrane at a scale of 10 μm, according to certain embodiments;

FIGS. 6A-6B show SEM/energy dispersive X-ray spectroscopy (EDX, EDS) analysis of the crosslinked PAN support, according to certain embodiments;

FIGS. 6C-6D show SEM/EDX analysis of the 4A-TPC@crosslinked PAN membrane, according to certain embodiments;

FIGS. 6E-6F show SEM/EDX analysis of the 4A-3P-TPC@crosslinked PAN membrane, according to certain embodiments;

FIG. 7A shows an EDS layered image of the crosslinked PAN support, according to certain embodiments;

FIG. 7B shows elemental mapping analysis depicting carbon distributed in the crosslinked PAN support, according to certain embodiments;

FIG. 7C shows elemental mapping analysis depicting nitrogen distributed in the crosslinked PAN support, according to certain embodiments;

FIG. 7D shows elemental mapping analysis depicting oxygen distributed in the crosslinked PAN support, according to certain embodiments;

FIG. 7E shows an EDS layered image of the 4A-TPC@crosslinked PAN membrane, according to certain embodiments;

FIG. 7F shows elemental mapping analysis depicting carbon distributed in the 4A-TPC@crosslinked PAN membrane, according to certain embodiments;

FIG. 7G shows elemental mapping analysis depicting nitrogen distributed in the 4A-TPC@crosslinked PAN membrane, according to certain embodiments;

FIG. 7H shows elemental mapping analysis depicting oxygen distributed in the 4A-TPC@crosslinked PAN membrane, according to certain embodiments;

FIG. 7I shows an EDS layered image of the 4A-3P-TPC@crosslinked PAN membrane, according to certain embodiments;

FIG. 7J shows elemental mapping analysis depicting carbon distributed in the 4A-3P-TPC@crosslinked PAN membrane, according to certain embodiments;

FIG. 7K shows elemental mapping analysis depicting nitrogen distributed in the 4A-3P-TPC@crosslinked PAN membrane, according to certain embodiments;

FIG. 7L shows elemental mapping analysis depicting oxygen distributed in the 4A-3P-TPC@crosslinked PAN membrane, according to certain embodiments;

FIG. 8 is a schematic illustration depicting the proposed structures of active layers 4A-TPC@crosslinked PAN membrane and 4A-3P-TPC@crosslinked PAN membrane, according to certain embodiments;

FIG. 9A shows water contact angle (WCA) for the crosslinked PAN support, according to certain embodiments;

FIG. 9B shows WCA for the 4A-TPC@crosslinked PAN membrane, according to certain embodiments;

FIG. 9C shows WCA for the 4A-3P-TPC@crosslinked PAN membrane, according to certain embodiments;

FIG. 10A is a 2D image depicting atomic force microscopy (AFM) results for the crosslinked PAN support, according to certain embodiments;

FIG. 10B is a 3D image depicting AFM results for the crosslinked PAN support, according to certain embodiments;

FIG. 10C is a 2D image depicting AFM results for the 4A-TPC@crosslinked PAN membrane, according to certain embodiments;

FIG. 10D is a 3D image depicting AFM results for the 4A-TPC@crosslinked PAN membrane, according to certain embodiments;

FIG. 10E is a 2D image depicting AFM results for the 4A-3P-TPC@crosslinked PAN membrane, according to certain embodiments;

FIG. 10F is a 3D image depicting AFM results for the 4A-3P-TPC@crosslinked PAN membrane, according to certain embodiments;

FIG. 11A depicts variation of permeate flux as a function of applied feed pressure for the 4A-TPC@crosslinked PAN membrane, according to certain embodiments;

FIG. 11B depicts variation of permeate flux as a function of applied feed pressure for the 4A-3P-TPC@crosslinked PAN membrane, according to certain embodiments;

FIG. 12 shows the effect of solvent viscosity on permeate flux for the 4A-TPC@crosslinked PAN membrane and the 4A-3P-TPC@crosslinked PAN membrane, according to certain embodiments;

FIG. 13A shows variation of permeate flux as a function of molecular weight of solutes (dyes) for the 4A-TPC@crosslinked PAN membrane and 4A-3P-TPC@crosslinked PAN membrane, according to certain embodiments;

FIG. 13B depicts rejection performance of the 4A-TPC@crosslinked PAN membrane and the 4A-3P-TPC@crosslinked PAN membrane for solutes (dyes), according to certain embodiments;

FIG. 14A depicts absorption spectra and coloration of feeds and permeates of Congo Red (CR) solute with the 4A-TPC@crosslinked PAN membrane, according to certain embodiments;

FIG. 14B depicts absorption spectra and coloration of feeds and permeates of Eriochrome black T (EBT) solute with the 4A-TPC@Crosslinked PAN membrane, according to certain embodiments;

FIG. 14C depicts absorption spectra and coloration of feeds and permeates of Methylene Blue (MB) solute for the 4A-TPC@crosslinked pan membrane, according to certain embodiments;

FIG. 15A shows absorption spectra and coloration of feeds and permeates of the CR solute with the 4A-3P-TPC@crosslinked PAN membrane, according to certain embodiments;

FIG. 15B shows absorption spectra and coloration of feeds and permeates of the EBT solute for the 4A-3P-TPC@crosslinked PAN membrane, according to certain embodiments; and

FIG. 15C shows absorption spectra and coloration of feeds and permeates of the MB solute for the 4A-3P-TPC@crosslinked PAN membrane, according to certain embodiments.

### DETAILED DESCRIPTION

In the following description, it is understood that other embodiments may be utilized, and structural and operational changes may be made without departure from the scope of the present embodiments disclosed herein.

Reference will now be made in detail to specific embodiments or features, examples of which are illustrated in the accompanying drawings. Whenever possible, corresponding or similar reference numbers will be used throughout the drawings to refer to the same or corresponding parts. Moreover, references to various elements described herein, are made collectively or individually when there may be more than one element of the same type. However, such references are merely exemplary in nature. It may be noted that any reference to elements in the singular may also be construed to relate to the plural and vice-versa without limiting the scope of the disclosure to the exact number or type of such elements unless set forth explicitly in the appended claims.

When describing the present disclosure, the terms used are to be construed in accordance with the following definitions, unless a context dictates otherwise.

Embodiments of the present invention will now be described more fully hereinafter with reference to the accompanying drawings wherever applicable, in that some, but not all embodiments of the disclosure are shown.

As used herein, the words “a,” “an,” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

As used herein, the term “nanoparticles” refers to particles having a particle size of 1 nanometer (nm) to 500 nm within the scope of the present disclosure. The nanoparticles (NPs) may exist in various morphological shapes, such as nanowires, nanocrystals, nanorectangles, nanotriangles, nanopentagons, nanohexagons, nanoprisms, nanorods, nanodisks, nanocubes, nanoribbons, nanoblocks, nanobeads, nanotoroids, nanodiscs, nanobarrels, nanogranules, nanowhiskers, nanoflakes, nanofoils, nanopowders, nanoboxes, nanostars, tetrapods, nanobelts, nano-urchins, nanoflowers, the like, and mixtures thereof.

As used herein, the term “porosity” refers to a measure of the void or vacant spaces within a material. Porosity is a ratio of the void or vacant space, sometimes referred to as pore volume, to a bulk volume. Porosity may be referred to as a fraction or a percentage.

As used herein, the term “particle size” refers to the length or longest dimension of a particle.

As used herein, the term “pore size” refers to the length or longest dimension of a pore opening.

As used herein, the term “membrane” refers to a porous structure that is capable of separating components of a homogeneous or heterogeneous fluid. A membrane may be a layer of varying thickness of semi-permeable material that may be used for solute separation as a transmembrane pressure is applied across the membrane. A degree of selectivity may be based on membrane composition, charge, and porosity. Membranes may have symmetric or asymmetric pores, wherein a membrane with asymmetric pores have variable pore diameters. Membranes may be used for microfiltration, ultrafiltration, nanofiltration, and reverse osmosis process. In particular, “pores” in the sense of the present disclosure indicate voids allowing fluid communication between different sides of the structure. Pores may have a varying pore size, pore size distribution, and pore morphology, such as pore shape and surface roughness. The pores may be made up of a network of interconnected channels. More particular in use when a homogeneous or heterogeneous fluid is passed through the membrane, some components of the fluid can pass through the pores of the membrane into a “permeate stream,” while some components of the fluid can be retained by the membrane and can thus accumulate in a “retentate,” and/or some components of the fluid can be rejected by the membrane into a “rejection stream.” The homogeneous or heterogeneous fluid that enters the membrane may be referred to herein as a “feed stream” or a “feed.” Membranes can be of various thicknesses, with homogeneous or heterogeneous structures. Membranes can be in the form of flat sheets or bundles of hollow fibers.

Membranes can also be in various configurations, including but not limited to spiral wound, tubular, hollow fiber, and other configurations identifiable to a skilled person upon a reading of the present disclosure. Membranes can also be classified according to their pore diameter. Membranes can be neutral or charged, and particle transport can be active or passive. The latter can be facilitated by pressure, concentration, and chemical or electrical gradients of the membrane process.

As used herein, “filtration” refers to the mechanical or physical operation or process that can be employed for the separation of constituents of homogeneous or heterogeneous solutions. Filtration may use a filter medium to separate components of homogeneous and heterogenous solutions. The filter medium may be a physical separator, such as a membrane, a chemical separator or gradient, an electrical separator or gradient, and any separator or gradient known in the art for separating solutions. Filtration may be used to separate solids from liquids, solids from gases, and/or liquids from other liquids. Filtration may be gravity-driven, pressure-driven, and/or vacuum-driven. Types of filtration can be categorized by the estimated sizes of materials to be separated and can involve particle filtration (>10 μm), microfiltration (0.1-10 μm), ultrafiltration (0.01-0.1 μm), nanofiltration (NF) (0.001-0.01 μm), reverse osmosis (RO) (<0.001 μm), and any filtration known in the art.

As used herein, “surface roughness” refers to a parameter establishing the performance of the membrane. Rougher surfaces may accumulate foulants in valleys, reducing permeate quality and flow rate.

As used herein, “water contact angle (WCA)” refers to a measure of the wettability of a solid surface. Hydrophobic solids have a contact angle above 90° (indicative of poor wetting), and hydrophilic solids have a contact angle below 90° (indicative of water-loving). The contact angle may be used for gauging the hydrophobicity, hydrophilicity, and/or extent of cleanliness of a surface.

As used herein, “surface charge” refers to an electric charge that exists on a two-dimensional (2D) surface. These electric charges are confined to this 2D surface, and the surface charge density, measured in coulombs per square meter (cm⁻²), is employed to explain the charge distribution on the surface.

As used herein, “water contact angle (WCA)” refers to a measure of the wettability of a solid surface. Hydrophobic solids have a contact angle above 90° (indicative of poor wetting), and hydrophilic solids have a contact angle below 90° (indicative of water-loving). The contact angle may be used for gauging the hydrophobicity, hydrophilicity, and/or extent of cleanliness of a surface.

As used herein, “pollutant” refers to a substance introduced into the environment (i.e., air, gas, water, liquid, and the like) that has undesired, or even detrimental, consequences.

As used herein, “flux” refers to a metric for comparing, scaling, and assessing the general performance of a membrane. It is defined as the permeate flow per unit of time and membrane surface area, commonly expressed as liters per square meter per hour (L m⁻² h⁻¹) (LMH).

Aspects of the present disclosure are directed toward the nanofiltration performance of polyamide (PA) thin film composite membranes, particularly organic solvent nanofiltration/solvent resistance nanofiltration (OSN/SRNF). Two preferable aliphatic amines with varying aliphatic chain lengths between primary and secondary amines are selected for this purpose. The two membranes, 4A-TPC@crosslinked PAN and 4A-3P@crosslinked PAN, were fabricated by using two different tetramines, 4A (N,N′-bis(3-aminopropyl)ethylenediamine) and 4A-3P (N,N′-Bis(2-aminoethyl)-1,3-propanediamine), crosslinked with terephthaloyl chloride (TPC) on a crosslinked polyacryonitrile (PAN) support through interfacial polymerization (IP). The presence of multiple hydrophobic —CH₂— groups in the structures of the aliphatic amines 4A and 4A-3P develops hydrophobic sites in the hydrophilic polyamide active layers of the membranes. In addition, 4A has two secondary amino groups separated by an ethylene (—CH₂—CH₂—) group, whereas, in 4A-3P, the two secondary amino groups are separated by propylene (—CH₂—CH₂—CH₂—), leading to variation in the structural features and performance of the two membranes. Both membranes were fully characterized by several membrane characterization techniques and applied for organic solvent nanofiltration/solvent resistance nanofiltration using both polar (methanol, ethanol, and isopropanol) and non-polar (n-hexane and toluene) solvents. Different dyes (Congo red, Eriochrome black T, and Methylene blue) were used as model solutes.

A filtration membrane is described. The filtration membrane, also referred to as a membrane, includes a thermoplastic substrate. It is desirable for the thermoplastic substrate to possess good mechanical and thermal properties. Also, the thermoplastic substrate should demonstrate high resistance to chemicals, such as aromatic hydrocarbons, ketones, ethers, and esters. The thermoplastic substrate is an aggregate material including a polymer component (thermoplastic polymer) configured to strengthen the membrane structure.

The thermoplastic substrate includes one or more thermoplastic polymers. Suitable examples of thermoplastic polymers include, but are not limited to, polypropylene (PP), polyamide (PA), polyacrylonitrile (PAN), polycarbonate (PC), high-density polyethylene (HDPE), low-density polyethylene (LDPE), polybutylene terephthalate (PBT), polyethylene terephthalate (PET), polyester terephthalate, polystyrene (PS), polyether ether ketone (PEEK), polyphenylene sulfide (PPS), polyetherimide (PEI), polyamide-imide (PAI), polyacrylic acid (PAA), acrylonitrile butadiene styrene (ABS), polylactic acid (PLA), polyvinyl chloride (PVC), polyurethanes (PUR), polyvinylidene fluoride (PVDF), polytetrafluoroethylene (Teflon), the like, and/or a mixture thereof. In some embodiments, blends of thermoplastic polymers may be used as well. In a preferred embodiment, the thermoplastic polymer includes a polyacrylonitrile.

In some embodiments, the thermoplastic substrate is in the form of interwoven fibers with a diameter of 2-20 micrometers (μm), preferably 3-19 μm, preferably 4-18 μm, preferably 5-17 μm, preferably 6-16 μm, preferably 7-15 μm, preferably 8-14 μm, preferably 9-13 μm, and preferably 10-12 μm. The interwoven fibers may be of varying diameter. The interwoven fibers may be in the form of a pattern, such as a tabby weave, a twill weave, or a satin weave, or may be in the form of a random pattern.

In some embodiments, the thermoplastic substrate has a thickness of 50-200 μm, preferably 60-190 μm, preferably 70-180 μm, preferably 80-170 μm, preferably 90-160 μm, preferably 100-150 μm, preferably 110-140 μm, and preferably 120-140 μm.

The membrane further includes a first layer including a PAN grafted with a diamine. In some embodiments, the first layer may further include copolymers of acrylonitrile. Suitable examples of co-polymers include, but are not limited to, styrene-acrylonitrile (SAN), acrylonitrile butadiene styrene (ABS), acrylonitrile styrene acrylate (ASA), acrylonitrile butadiene (NBR), modified polymers thereof, or the like. Suitable examples of diamines include, but are not limited to, hydrazine, piperazine (PIP), m-phenylenediamine (MPD), and ethylene diamine (EDA). In a preferred embodiment, the diamine is a hydrazine, preferably hydrazine.

In some embodiments, other polymers such as polyvinyl chloride, polyvinylidene chloride, polyacrylonitrile, high impact PS, ABS, polysulfone, polyethylene/acrylonitrile butadiene styrene, polycarbonate/acrylonitrile butadiene styrene, polybutadiene, polyisoprene, polyacetylene, silicones, synthetic rubbers, the like, and copolymers and mixtures thereof may be used as well in addition to and/or instead of PAN. In some embodiments, other diamines such as ethylene diamine, 1,3-diaminopropane, 1,4-diaminobutane, hexamethylenediamine, and the like may be used as well in addition to and/or hydrazine.

In some embodiments, the first layer includes crosslinked PAN in the form of vertical hollow tubes having a diameter of 1-50 μm, preferably 2-49 μm, preferably 3-48 μm, preferably 4-47 μm, preferably 5-46 μm, preferably 6-45 μm, preferably 7-44 μm, preferably 8-43 μm, preferably 9-42 μm, preferably 10-41 μm, preferably 11-40 μm, preferably 12-39 μm, preferably 13-38 μm, preferably 14-37 μm, preferably 15-36 μm, preferably 16-35 μm, preferably 17-34 μm, preferably 18-33 μm, preferably 19-32 μm, preferably 20-31 μm, preferably 21-30 μm, preferably 22-29 μm, preferably 23-28 μm, preferably 24-27 μm, and preferably 25-26 μm. In some embodiment, the crosslinked PAN in the form of vertical hollow tubes having a length of 2-100 μm, preferably 5-95 μm, preferably 10-90 μm, preferably 15-85 μm, preferably 20-80 μm, preferably 25-75 μm, preferably 30-70 μm, preferably 35-65 μm, preferably 40-60 μm, and preferably 45-55 μm. In some embodiments, the first layer has a thickness at least 20% less than the thickness of the thermoplastic substrate, preferably 20-30%, 30-40%, 40-50%, 50-75%, or 75-95% less than the thickness of the thermoplastic substrate.

The membrane further includes a second layer, including reacted units of a tetramine and reacted units of a phthaloyl chloride. A chemical compound that consists of four amine groups is commonly referred to as tetramine. In some embodiments, the tetramine has two secondary amine groups and two primary amine groups. The primary amine groups are separated from the secondary amine groups by a C2 (two carbon) or C3 (three carbon) aliphatic group. In a preferred embodiment, the tetramine includes two primary amines and two secondary amines. The tetramine may be N,N′-Bis(2-aminoethyl)-1,3-propanediamine (4A-3P), N,N′-bis(3-aminopropyl)ethylenediamine (4A), or a mixture thereof. In a preferred embodiment, the tetramine is 4A-3P. In another embodiment, the tetramine is 4A. In a preferred embodiment, the phthaloyl chloride is TPC, which acts as a cross-linker. In some embodiments, the reacted units of tetramine and the reacted units of phthaloyl chloride bond through both the primary and secondary amino groups of the tetramine. In an embodiment, a primary amine of a first tetramine is covalently cross-linked with a primary amine of a second tetramine with reacted units of phthaloyl chloride. In an embodiment, a secondary amine of a first tetramine is covalently cross-linked with a secondary amine of a second tetramine with reacted units of phthaloyl chloride. The tetramine covalently cross-links with the TPC during the interfacial polymerization (IP) process to form a polyamide (PA). In some embodiments, one or more amine groups of the crosslinked PAN of the first layer are covalently bonded to the second layer through one or more phthaloyl groups of the reacted units of the phthaloyl chloride. In some embodiments, one or more amine groups of the crosslinked PAN of the first layer are covalently crosslinked to a tetramine of the second layer through one or more phthaloyl groups of the reacted units of the phthaloyl chloride. In some embodiments, one or more amine groups of the crosslinked PAN of the first layer are covalently crosslinked to a primary amine and/or a secondary amine of the tetramine of the second layer through one or more phthaloyl groups of the reacted units of the phthaloyl chloride. In some embodiments, the reacted units of phthaloyl chloride may facilitate bonding between the first layer and the second layer. In some embodiments, amines from the PAN grafted with the diamine of the first layer may be covalently bonded to the tetramines of the second layer. The membranes were fabricated through an interfacial polymerization reaction on crosslinked PAN support.

In some embodiments, the second layer is in the form of nanoparticles, preferably a continuous layer of nanoparticles, with a diameter of 2-500 nm, 5-470 nm, preferably 10-450 nm, preferably 20-430 nm, preferably 40-400 nm, preferably 50-370 nm, preferably 70-350 nm, preferably 90-320 nm, preferably 100-300 nm, preferably 120-270 nm, preferably 140-260 nm, preferably 150-250 nm, preferably 170-230 nm, or preferably 180-220 nm.

In some embodiments, the second layer has a thickness of 900-1000 nm, preferably 905-995 nm, preferably 910-990 nm, preferably 915-985 nm, preferably 920-980 nm, preferably 925-975 nm, preferably 930-970 nm, preferably 935-965 nm, preferably 940-960 nm, and preferably 945-955 nm. In a preferred embodiment, the second layer has a thickness of about 937 nm. In another embodiment, the second layer has a thickness of about 958.1 nm.

In some embodiments, the membrane has a WCA of 55-70°, preferably 56-69°, preferably 57-68°, preferably 58-67°, preferably 59-66°, preferably 60-65°, preferably 61-64°, and preferably 62-63°. In a preferred embodiment, the membrane has a WCA of about 59.9°. In another embodiment, the membrane has a WCA of about 65.1°.

In some embodiments, the membrane has an average roughness of 10-30 nm, preferably 11-29 nm, preferably 12-28 nm, preferably 13-27 nm, preferably 14-26 nm, preferably 15-25 nm, preferably 16-24 nm, preferably 17-23 nm, preferably 18-22 nm, and preferably 19-21 nm. In a preferred embodiment, the membrane has an average roughness of about 12.8 nm. In another embodiment, the membrane has an average roughness of about 28.7 nm.

In some embodiments, the membrane has a surface charge of −0.75 to −5.50 mV, preferably −1 to −5.25 mV, preferably −1.25 to −5 mV, preferably −1.5 to −4.75 mV, preferably −1.75 to −4.5 mV, preferably −2 to −4.25 mV, preferably −2.25 to −4 mV, preferably −2.5 to −3.75 mV, and preferably −2.75 to −3.5 mV. In a preferred embodiment, the membrane has a surface charge of −0.937 mV. In another embodiment, the membrane has a surface charge of −5.25 mV.

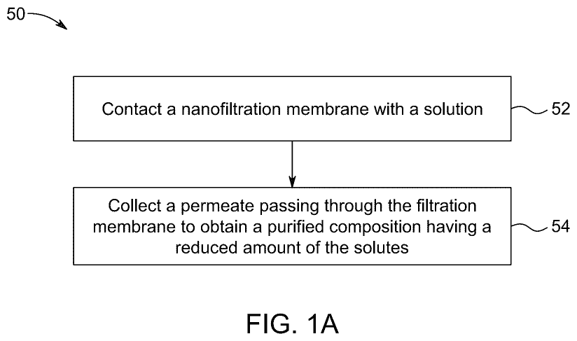

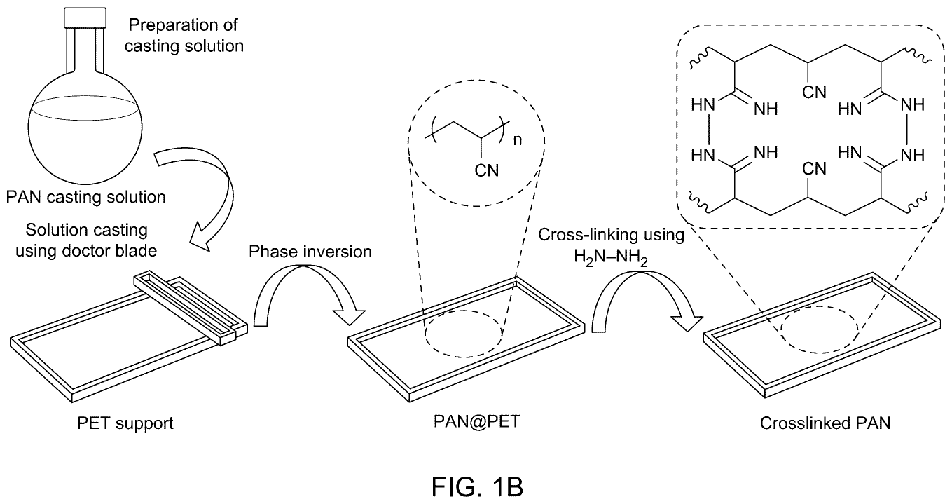

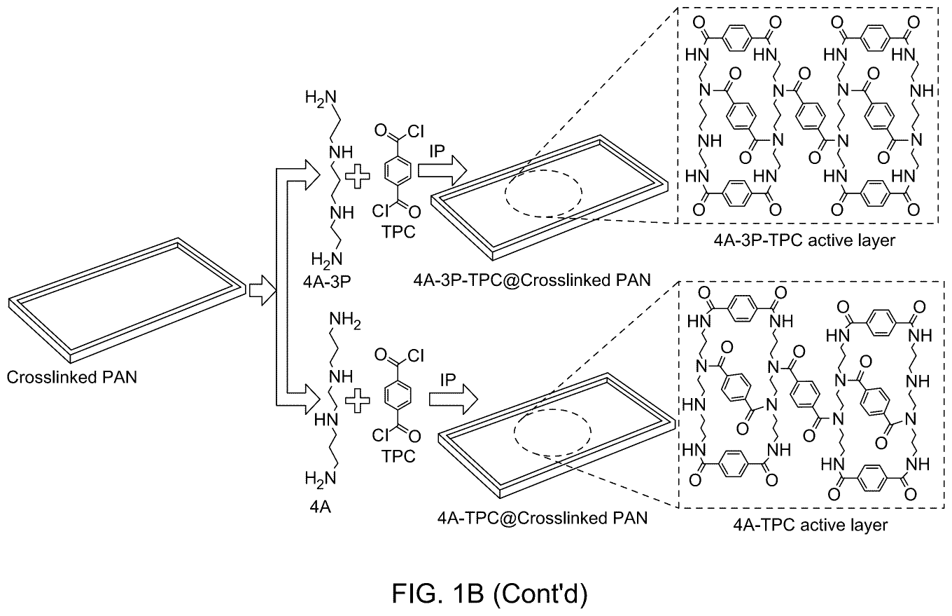

FIG. 1A illustrates a flow chart of a method of nanofiltration. The order in which the method 50 is described is not intended to be construed as a limitation, and any number of the described method steps can be combined in any order to implement the method 50. Additionally, individual steps may be removed or skipped from the method 50 without departing from the spirit and scope of the present disclosure.

At step 52, the method 50 includes contacting the nanofiltration membrane with a solution. The solution involves one or more polar solvents, one or more non-polar solvents, and one or more solutes. Polar solvents are solvents containing partial positive and partial negative charge. Suitable examples of polar solvents include water, methanol, ethanol, acetone, dimethyl sulfoxide (DMSO), dimethylformamide, dimethylacetamide, and isopropanol. In a preferred embodiment, the polar solvents include methanol, ethanol, and isopropanol. Non-polar solvents are solvents that preferably do not contain partial positive and partial negative charge. Polar compounds may not be soluble or miscible by non-polar solvents, but hydrophobic chemicals can be dissolved by them. Suitable examples of non-polar solvents include benzene, hexane, carbon tetrachloride, toluene, diethyl ether, and chloroform. In a preferred embodiment, the non-polar solvents include hexane and toluene. A solute is a substance dissolved in a solution and the amount of solute is less than the amount of solvent. In some embodiments, the solute is a pollutant. In some embodiments, the pollutants are selected from a group consisting of methylene blue (MB), eriochrome black T (EBT), and Congo red (CR).

At step 54, the method 50 includes collecting a permeate passing through the filtration membrane to obtain a purified composition having a reduced amount of the solutes. In some embodiments, the membrane has a permeate flux of 1-80 L m⁻² h⁻¹, preferably 5-75 L m⁻² h⁻¹, preferably 10-70 L m⁻² h⁻¹, preferably 15-65 L m⁻² h⁻¹, preferably 20-60 L m⁻² h⁻¹, preferably 25-55 L m⁻² h⁻¹, preferably 30-50 L m⁻² h⁻¹, preferably 35-45 L m⁻² h⁻¹ at a pressure of 4 bar. In some embodiments, when the polar solvent is methanol, the membrane has a permeate flux of 7-13 L m⁻² h⁻¹, preferably 8-12 L m⁻² h⁻¹, and preferably 9-11 L m⁻² h⁻¹ at a pressure of 4 bar. In a preferred embodiment, when the polar solvent is methanol, the membrane has a permeate flux of 8.2 L m⁻² h⁻¹ at a pressure of 4 bar. In another embodiment, when the polar solvent is methanol, the membrane has a permeate flux of 12 L m⁻² h⁻¹ at a pressure of 4 bar.

In some embodiments, when the polar solvent is methanol, the membrane has a rejection percentage of the pollutants of 4-100 weight percent (wt. %), 5-95 wt. %, 10-90 wt. %, 15-85 wt. %, 20-80 wt. %, 25-75 wt. %, 30-70 wt. %, 35-65 wt. %, 40-60 wt. %, and 45-55 wt. % based on an initial weight of the pollutants. In some embodiments, when the pollutant is EBT, the membrane has a rejection percentage of the EBT of 93-100 wt. %, 94-99 wt. %, 95-98 wt. %, and 96-97 wt. % based on an initial weight of the EBT. In a preferred embodiment, when the pollutant is EBT, the membrane has a rejection percentage of the EBT of 99.1 wt. %. In another embodiment, when the pollutant is EBT, the membrane has a rejection percentage of the EBT of 94.4 wt. %. In some embodiments, when the pollutant is CR, the membrane has a rejection percentage of the CR of 98-100 wt. %, preferably 98.5-99.5 wt. %, based on an initial weight of the CR. In a preferred embodiment, when the pollutant is CR, the membrane has a rejection percentage of the CR of 99.1 wt. %. In another embodiment, when the pollutant is CR, the membrane has a rejection percentage of the CR of 98.8 wt. %.

### EXAMPLES

The following examples describe and demonstrate exemplary embodiments of a filtration membrane as described herein. The examples are provided solely for the purpose of illustration and are not to be construed as limitations of the present disclosure, as many variations thereof are possible without departing from the spirit and scope of the present disclosure.

### Example 1: Materials

Terephthaloyl chloride (TPC; ≥99%), N,N′-Bis(2-aminoethyl)-1,3-propanediamine (4A-3P; 97%), N,N′-bis(3-aminopropyl)ethylenediamine (4A; 90%), triethylamine (TEA; ≥99.5%), polyacrylonitrile (PAN; ≥99%; average molecular weight=150,000), n-hexane (≥99%), N,N′-dimethyl formamide (DMF, ≥99%), polyethylene terephthalate (PET) fabric, methylene blue (MB; ≥99%), Eriochrome black T (EBT; ≥99%), and Congo red (CR; ≥99%) were all purchased from Sigma (St. Louis, USA). Deionized (DI) water from the in-lab setup was used.

### Example 2: Fabrication of PAN Support and Crosslinking of PAN Support

The PAN dope solution was prepared by dissolving 12 g of dried PAN in 88 g of DMF. The solution was stirred at room temperature overnight, leading to a transparent solution of PAN (PAN dope solution). The PAN dope solution was allowed to stand until all the air bubbles were removed. The PAN support was prepared by spreading an appropriate amount of PAN dope solution on a PET sheet affixed to a glass plate. Then the PAN dope solution was cast using a custom-made Doctor's blade (100 μm slit width) on the PET support. Immediately after casting PAN, the support was dipped in a deionized water bath and allowed to stand, leading to phase inversion. Hence, PAN/PET ultrafiltration support (also referred to as PAN@PET and/or PAN support) was fabricated. The PAN support was crosslinked by dipping an appropriate piece of PAN/PET support in a 25% (v/v) hydrazine (NH₂—NH₂) aqueous solution at 70° C. for 6 hours. Then, the crosslinked PAN support was thoroughly washed with an excess of DI water.

### Example 3: Fabrication of Membranes Through Interfacial Polymerization (IP)

Referring to FIG. 1B, a schematic diagram depicting the different stages involved in the fabrication of crosslinked PAN membranes is illustrated. The membranes were fabricated through an IP reaction on the crosslinked PAN support. The crosslinked PAN support was dipped in an aqueous solution of either amine, 4A-3P or 4A, for 10 minutes with continuous shaking on a seesaw shaker. The amine-impregnated crosslinked PAN support was taken out of the aqueous amine solution, and the excess amine was removed by using a rubber roller. Then amine impregnated crosslinked PAN support was dipped in a 0.15% (w/v) n-hexane solution of TPC for 60 seconds. The membrane was removed from the TPC solution and washed with fresh n-hexane to remove unreacted TPC from the membrane surface.

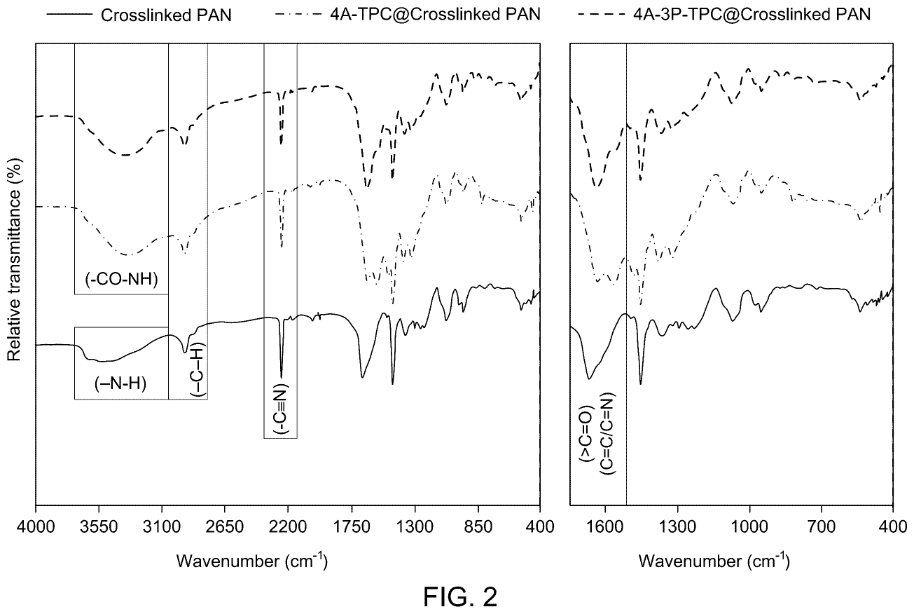

Referring to FIG. 2, the attenuated total reflection-Fourier transform infrared (ATR-FTIR) spectra of different crosslinked PAN membranes is illustrated. As can be seen from FIG. 2, to establish and understand the structure of the different membranes, namely crosslinked PAN, 4A-TPC@crosslinked PAN, and 4A-3P-TPC@crosslinked PAN were characterized by ATR-FTIR. The anticipated functional groups have been identified in different membranes. The FTIR spectra of the different membranes contain a broad peak in a range of 3600 cm⁻¹ to 3200 cm⁻¹, which can be attributed to the stretching vibration of the N—H bond of the amide linkage (—CO—NH) of the polyamide active layer of 4A-TPC@crosslinked PAN and 4A-3P-TPC@crosslinked PAN. Similarly, in the case of crosslinked PAN, the 3600 cm⁻¹ to 3200 cm⁻¹ broad peak can be attributed to the existence of the —N—H bond generated during crosslinking using hydrazine (NH₂—NH₂) as a crosslinking agent. Further, as can be seen in FIG. 2, a short but relatively sharp peak is located at 2900 cm⁻¹ to 2800 cm⁻¹ in each of the FTIR spectra of the membranes, including crosslinked PAN. This characteristic peak is due to —C—H stretching of the aliphatic backbones of the polyamide active layer due to constituent amines 4A, 4A-3P, and PAN support. Furthermore, a sharp and deep peak can be seen from FIG. 2 at around 2200 cm⁻¹ which is due to residual nitrile (—C≡N) groups in PAN. A relatively medium intensity peak of crosslinked PAN can be seen in the region of 1700 cm⁻¹ to 1650 cm⁻¹ which can be attributed to the stretching vibration of C═C/C═N bonds that originated during the crosslinking event of PAN with hydrazine. However, in the case of 4A-TPC@crosslinked PAN and 4A-3P-TPC@ crosslinked PAN membranes, a doublet peak is shown in the region of 1700 cm⁻¹ to 1600 cm⁻¹. In addition to the C═C/C—N bonds of PAN, 4A-TPC@crosslinked PAN and 4A-3P-TPC@crosslinked PAN membranes have carbonyl functional group (>C═O) of the primary/secondary amide linkages of polyamide active layers. The doublet in the region of 1700 cm⁻¹ to 1600 cm⁻¹ is attributed to the C═C/C═N and >C═O bonds of PAN and polyamide active layers. Therefore, the ATR-FTIR spectra of crosslinked PAN, 4A-TPC@crosslinked PAN, and 4A-3P-TPC@crosslinked PAN membranes have confirmed the presence of the functional groups and bonds in their structures, as shown in FIG. 1B.

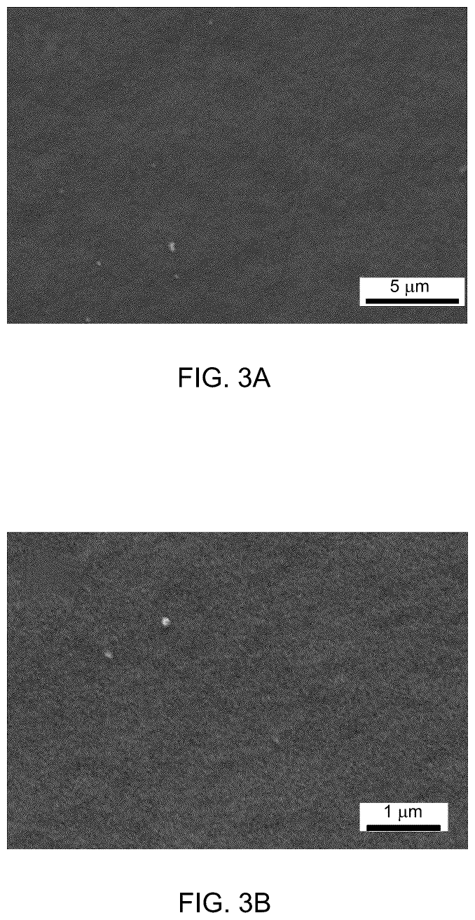

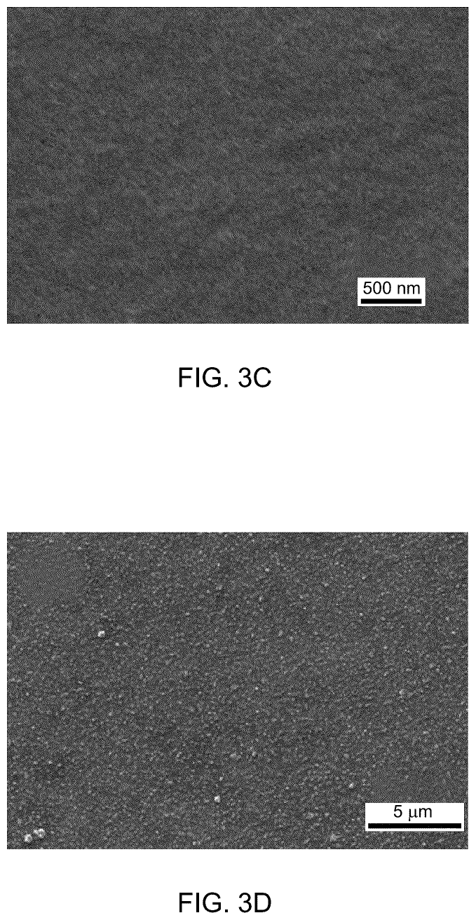

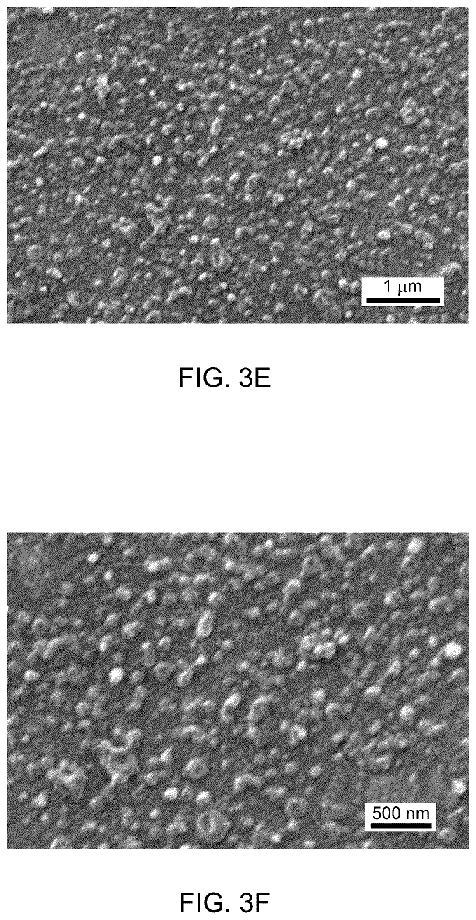

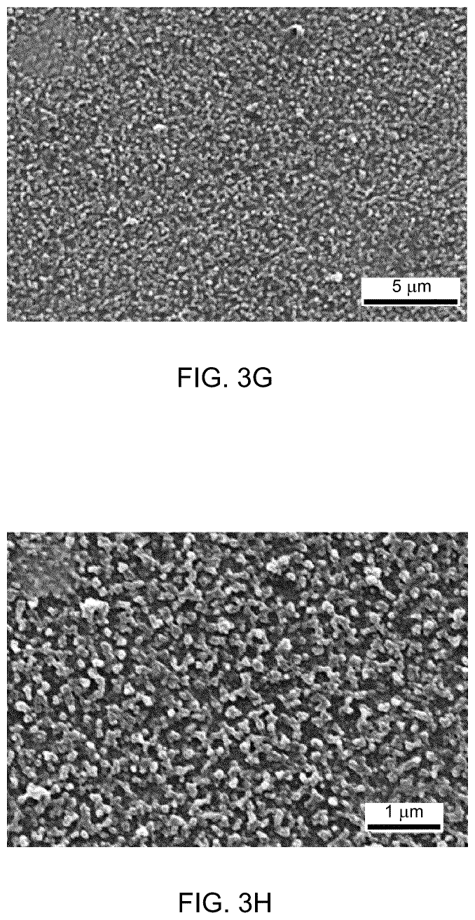

The membranes' surface morphology was investigated by studying and analyzing the SEM micrographs of the crosslinked PAN support, 4A-TPC@crosslinked PAN, and 4A-3P-TPC@crosslinked PAN membranes. Referring to FIGS. 3A-3C, scanning electron microscopy (SEM) micrographs of crosslinked PAN support are illustrated. As can be seen from FIGS. 3A-3C, the crosslinked PAN support appeared highly porous with a uniform sponge-like structure. After IP, the membrane surface appeared to be considerably rougher owing to the growth of a polyamide active layer on the support. Referring to FIGS. 3D-3F, SEM micrographs of 4A-TPC@crosslinked PAN membrane is illustrated. In the case of 4A-TPC@crosslinked PAN, the polyamide active layer appeared beaded, with the beads embedded in the matrix of polyamide. The geometry of the 4A-TPC@crosslinked PAN membrane resembles ridge and valley structure. However, the ridge and valley conformation are not perfectly adopted by the 4A-TPC@crosslinked PAN membrane as the valleys are filled with a polyamide network. Referring to FIGS. 3G-3I, SEM micrographs of the 4A-3P-TPC@crosslinked PAN membrane is illustrated. In the case of the 4A-3P-TPC@crosslinked PAN membrane, the polyamide active layer matches the traditional ridge and valley configuration of the polyamide membranes. In some embodiments, the variation in the pattern of growth of the polyamide active layer on PAN support can be attributed to the change in the structure of tetramine reacting during IP with the same crosslinker TPC. In the case of 4A-3P, secondary amine (—NH—) groups are located at the ends of propyl chain (3 carbon atoms apart), and in the case of 4A, the secondary amine (—NH—) groups are separated by ethyl chain (2 carbon atoms apart). The longer chain length provides a certain degree of freedom for penetration of TPC, leading to extensive crosslinking and the formation of ridge and valley patterns, as in the case of 4A-3P. In the case of 4A, the amine groups are comparatively close together, offering less chance for TPC to penetrate. Hence, the 4A-3P-TPC@crosslinked PAN membrane possesses a geometry for the efficient transfer or permeation of solvent molecules, while rejecting the solutes. The chemistry of the reacting monomers is a factor in tuning the performance of the nanofiltration membranes.

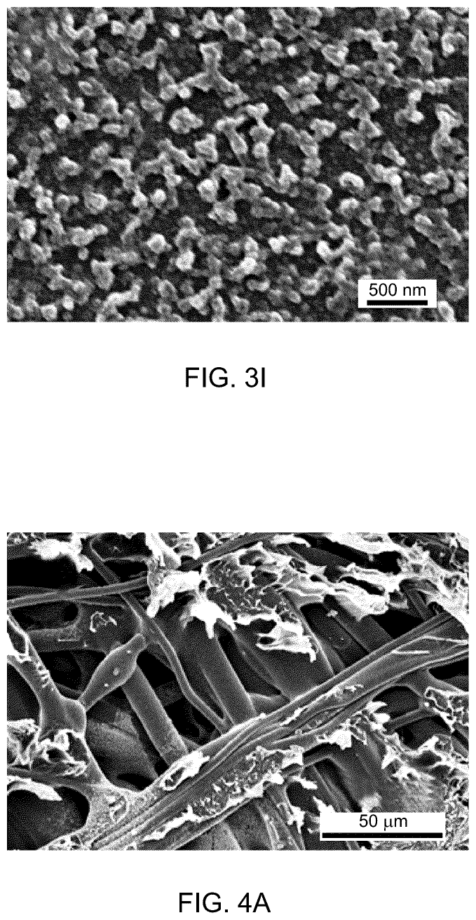

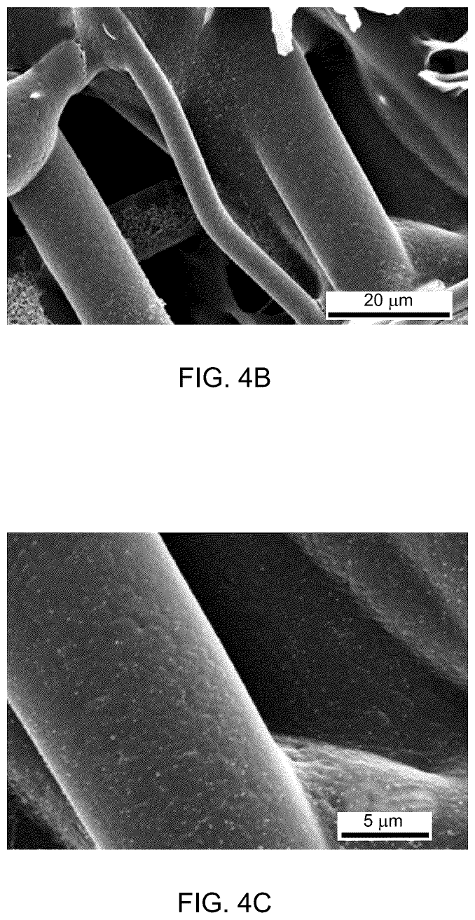

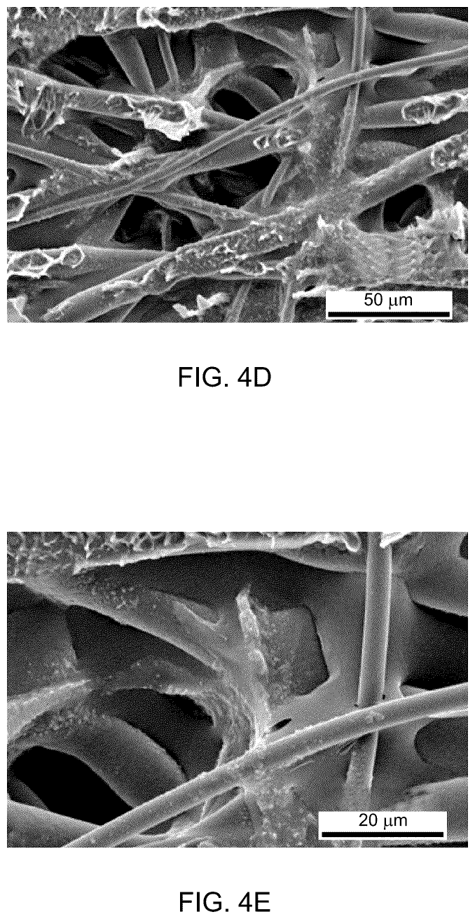

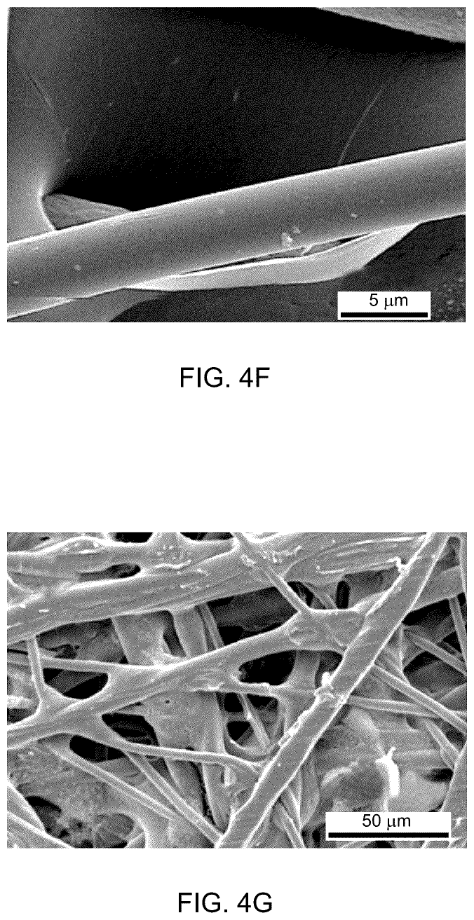

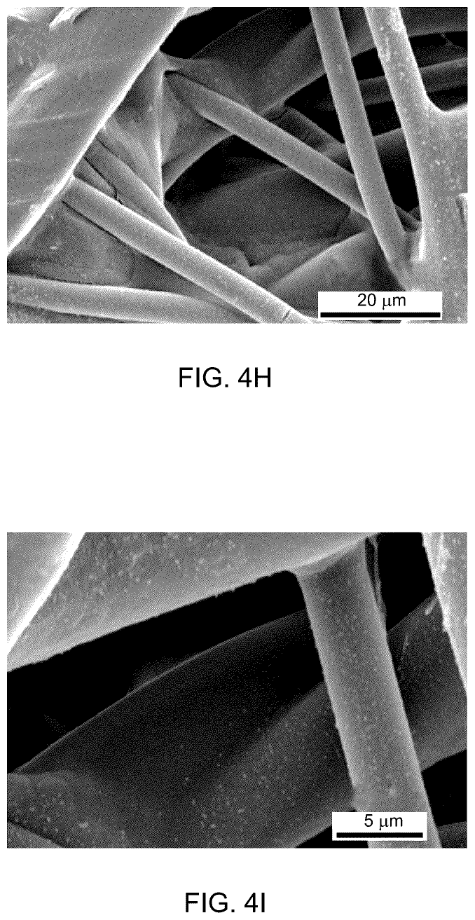

SEM micrographs of the backsides of the PAN/PET support are illustrated in FIGS. 4A-4C, SEM micrographs of the backsides of 4A-TPC@crosslinked PAN membrane are illustrated in FIGS. 4D-4F, and SEM micrographs of the backsides of 4A-3P-TPC@crosslinked PAN membrane are illustrated in FIGS. 4E-4I. The morphological analysis of the micrographs revealed that there was no growth of an active layer on the backside of the PET membrane. This might be attributed to the highly porous nature of the PET support, which is not able to retain a large amount of amine monomer required for IP after dipping in an n-hexane solution of TPC. Hence, the highly porous nature of the support is required to provide free passage to the solvents during filtration experiments.

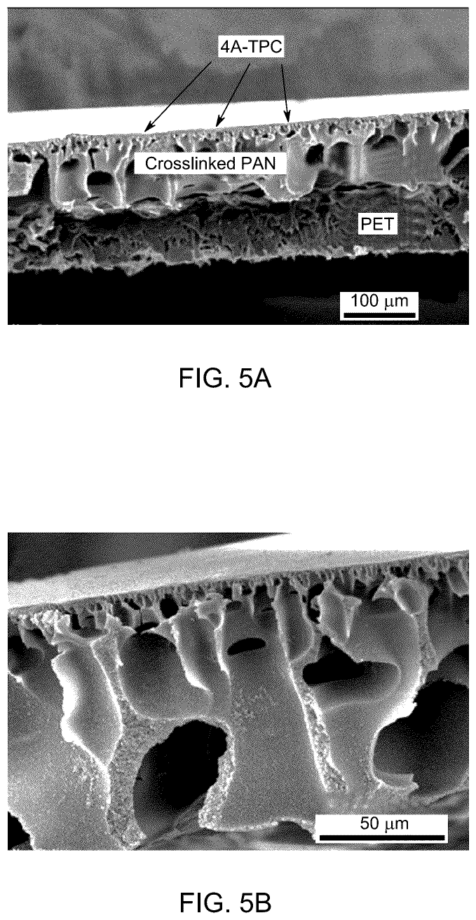

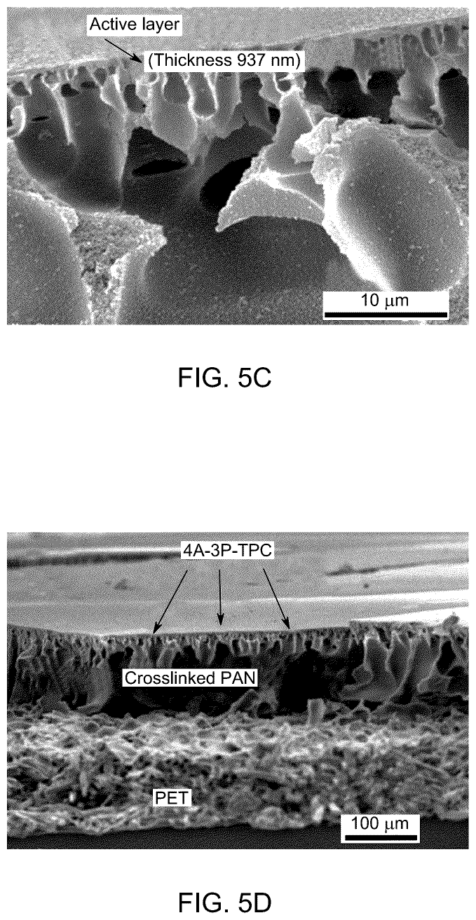

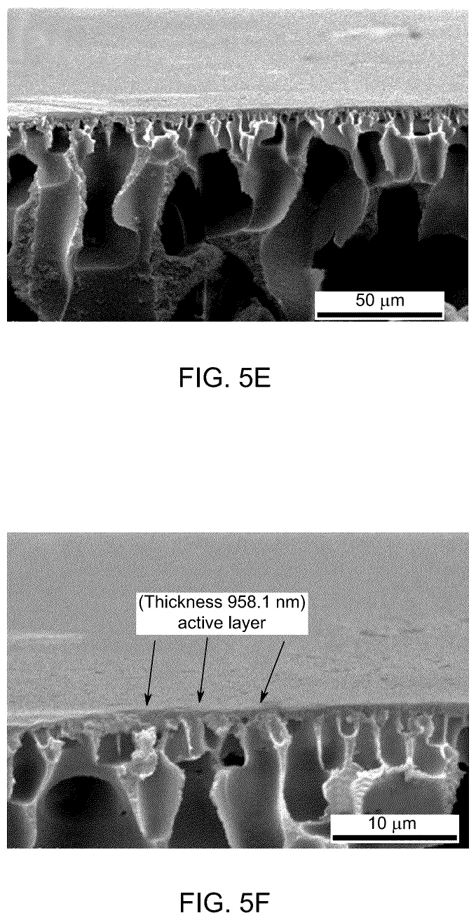

The SEM micrographs of cross sections of the 4A-TPC@crosslinked membrane are illustrated in FIGS. 5A-5C and the SEM micrographs of cross sections of the 4A-3P-TPC@crosslinked membrane are illustrated in FIGS. 5D-5F. As a collective result, it can be observed from FIGS. 5A-5F (for both membranes), the cross sections showed the presence of all three layers of the TFC membrane, which include highly porous and fibrous unwoven PET at the bottom of the membrane. Further, the middle layer was the ultrafiltration PAN support showing uniformly distributed finger-like projections, which indicates the PAN/PET support provides a support with minimum mass transfer resistance during filtration experiments. The third layer was the highly dense and skin-like polyamide selective layer with a thickness of 937 nm for 4A-TPC@crosslinked PAN and 958.1 nm for 4A-3P-TPC@crosslinked PAN membranes. Hence, the cross-sectional SEM micrographs revealed a characteristically asymmetric TFC membrane structure, which contributes to the separation capability of the TFC membranes.

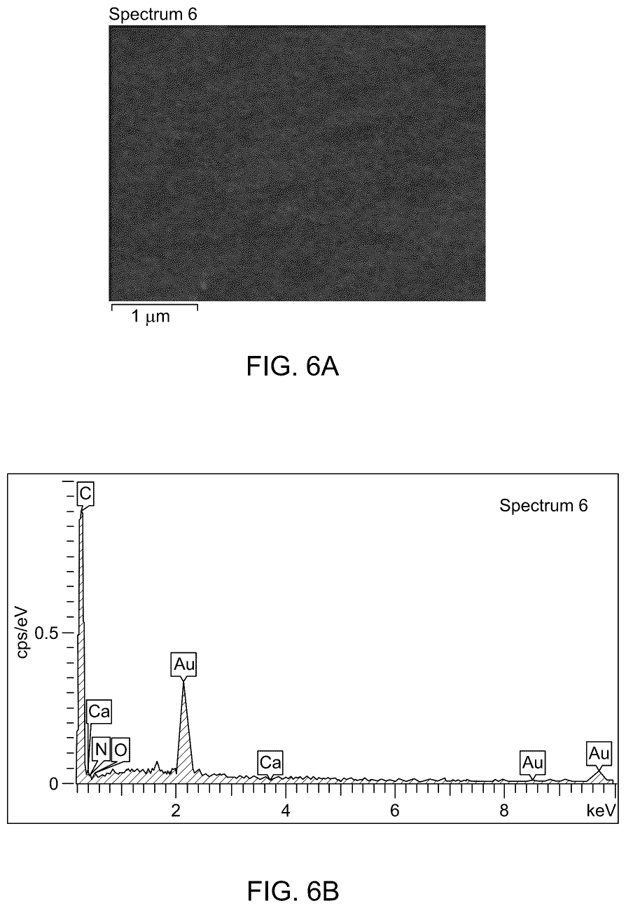

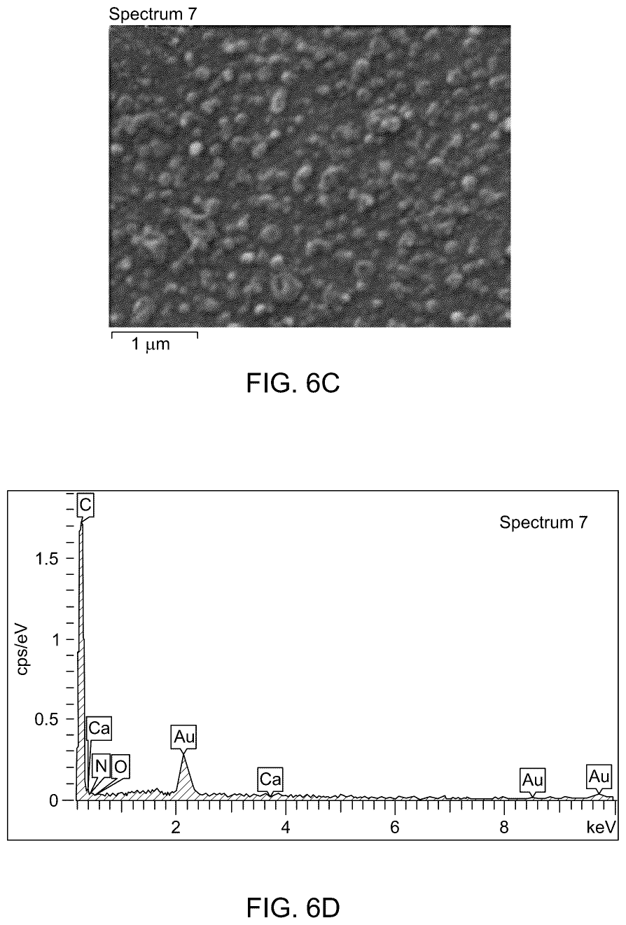

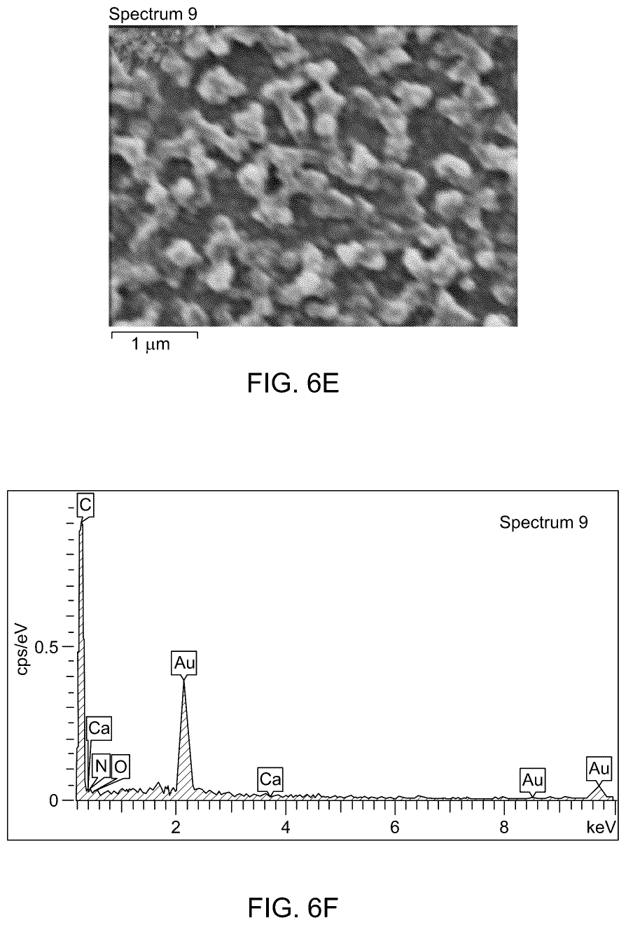

EDX analysis was carried out to identify the elemental composition of the PAN support (FIGS. 6A-6B), 4A-TPC@crosslinked PAN membrane (FIGS. 6C-6D), and 4A-3P-TPC@crosslinked PAN membrane (FIGS. 6E-6F). As can be seen from FIGS. 6A-6F, the EDX analysis of a selected area showed the presence of the elements (C, N, and O) that were present in contributing amines, TPC, PAN, and hydrazine used during membrane fabrication.

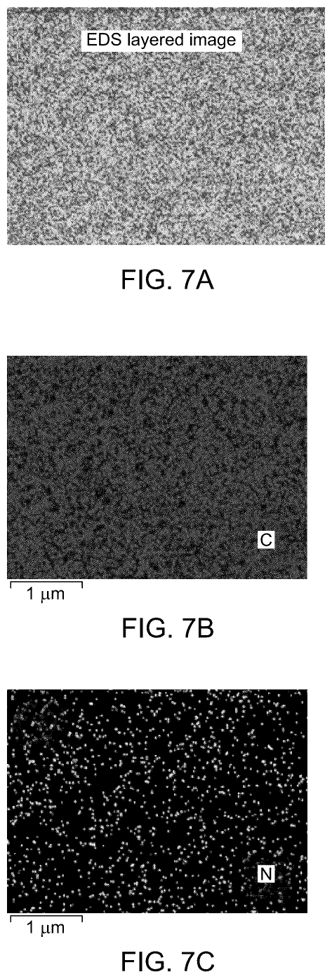

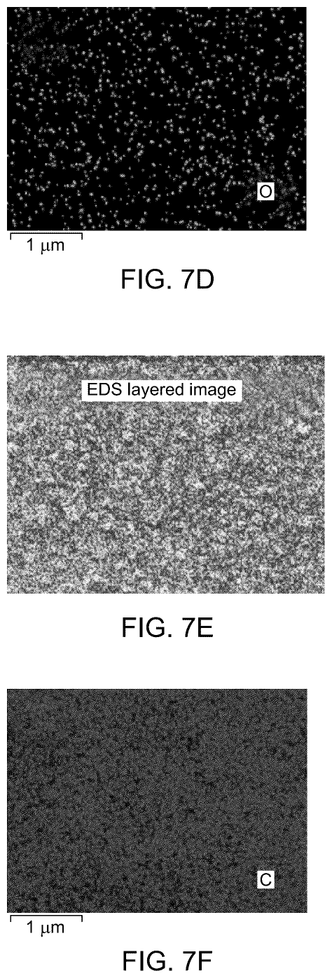

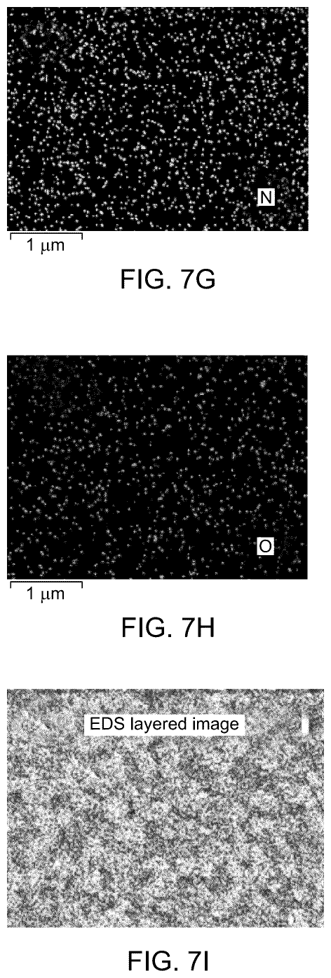

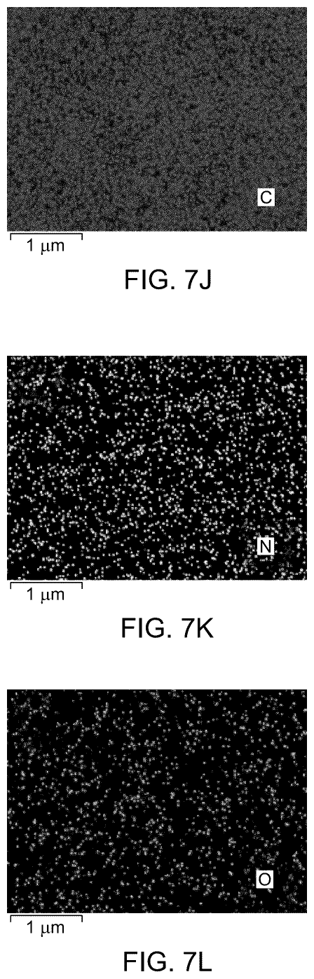

Referring to FIGS. 7A-7D, elemental mapping analysis of crosslinked PAN support is illustrated. As can be seen from FIGS. 7A-7D, the elemental mapping analysis revealed a uniform distribution of the elements (C, N, and O) throughout the entire crosslinked PAN support. Referring to FIGS. 7E-7H, elemental mapping analysis of 4A-TPC@crosslinked membrane is illustrated. As can be seen from FIGS. 7E-7H, the elemental mapping analysis revealed a uniform distribution of the elements (C, N, and O) throughout the entire 4A-TPC@crosslinked membrane. Referring to FIGS. 71-7L, elemental mapping analysis of 4A-3P-TPC@crosslinked membrane is illustrated. As can be seen from FIGS. 71-7L, the elemental mapping analysis revealed a uniform distribution of the elements (C, N, and O) throughout the entire 4A-3P-TPC@crosslinked membrane.

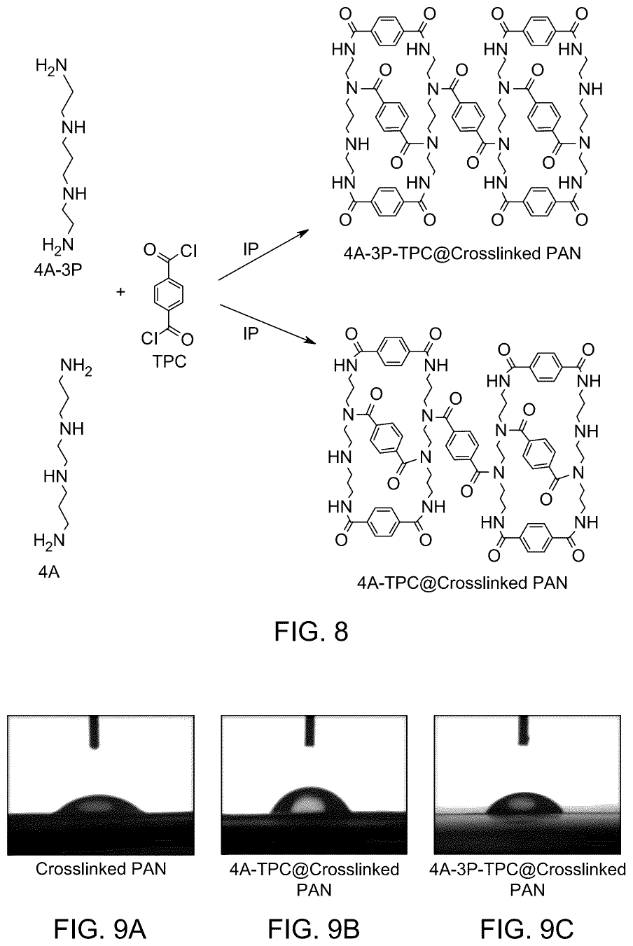

Referring to FIG. 8, a schematic representation depicting the proposed structure of active layers of the fabricated membranes is illustrated. As observed in FIG. 8, the polyamide active layers are generated because of a reaction between tetramines (4A and 4A-3P) and terephthalyol chloride (TPC). The 4A and 4A-3P amines have closely resembling structures with differences in the position of amino groups in the structure of tetramines. In the case of 4A-3P amine, the two secondary amino groups (—NH—) are separated by a propyl chain, while the primary amino groups (—NH₂) are separated from the secondary amino groups (—NH—) by ethyl chains. In particular, the structure of 4A-3P allows flexibility of binding of TPC with the neighboring 4A-3P chains. In the case of 4A amine, the secondary amino groups (—NH—) are separated by ethyl chains, and the primary amino groups (—NH₂) are separated from the secondary amino groups (—NH—) by propyl chains. As the secondary amino groups are close together in the 4A amine, the incorporation of TPC in between the amines is not flexible and uniform. Therefore, the 4A-3P amine leads to the formation of a network with more uniform crosslinking in the polyamide active layers. The SEM analysis (as illustrated in FIGS. 3G-31) confirmed the growth of more extensive crosslinking in the polyamide active layer of the membrane.

Referring to FIGS. 9A-9C, water contact angles (WCAs) for the crosslinked PAN support, 4A-TPC@crosslinked PAN membrane, and 4A-3P-TPC@crosslinked PAN membrane, respectively, is depicted. The surface hydrophilicity of the crosslinked PAN support and the PAN membranes was determined by recording the WCA. It was observed that the WCA of the hydrazine-crosslinked PAN support was found to be 45.9°, and hence, the crosslinked PAN support was hydrophilic. The hydrophilicity of crosslinked PAN support can be attributed to the contribution of hydrazine introducing amino groups in the support. Further, the hydrophilicity of crosslinked PAN can also be due to partial hydrolysis of the PAN support. After the growth of the polyamide active layer on crosslinked PAN support, the membrane surface hydrophilicity decreased as the WCA increased to 65.1° in the case of 4A-TPC@crosslinked PAN membrane and 59.9° for 4A-3P-TPC@crosslinked PAN. This might be attributed to the covering of highly hydrophilic crosslinked PAN support. In addition, the inclusion of phenyl rings of TPC in the polyamide active layer can also contribute to increasing the value of the WCA and decreasing the surface hydrophilicity of the membrane.

Although the WCA of 4A-TPC@crosslinked PAN and 4A-3P-TPC@crosslinked PAN membranes were slightly increased, the values of WCAs are still below 90°. Hence, both 4A-TPC@crosslinked PAN and 4A-3P-TPC@crosslinked PAN membranes are hydrophilic. The WCA of a surface depends upon surface roughness and chemical composition, as well as other factors. Generally, WCA decreases with a decrease in surface roughness, increasing the hydrophilicity of the membrane or vice versa. Compared to pristine crosslinked PAN support, the surface roughness of the fabricated membranes was increased. The average surface roughness was increased from 5.35 nm in the PAN support to 12.8 nm in the case of 4A-TPC@crosslinked PAN. In the case of 4A-3P-TPC@crosslinked PAN, the roughness was increased to 28.7 nm. Therefore, the surface roughness, as well as the WCAs, of 4A-TPC@crosslinked PAN and 4A-3P-TPC@crosslinked PAN were also increased in comparison to the crosslinked PAN support. Among the fabricated membranes, 4A-3P-TPC@crosslinked PAN has the highest surface roughness, but the WCA of 4A-3P-TPC@crosslinked PAN is slightly lower compared to 4A-TPC@crosslinked PAN. In addition to the membrane surface roughness, surface chemistry also plays a role in contributing to the hydrophilicity of the membranes. The presence of residual amino groups in the active layers of the membrane along with —COOH groups may contribute to the overall hydrophilicity of the membranes.

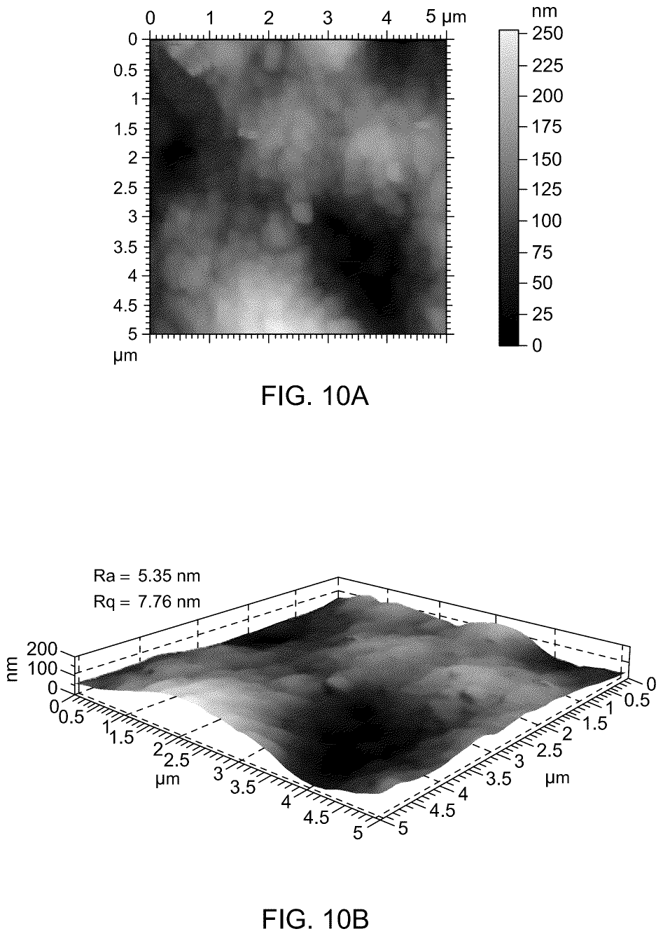

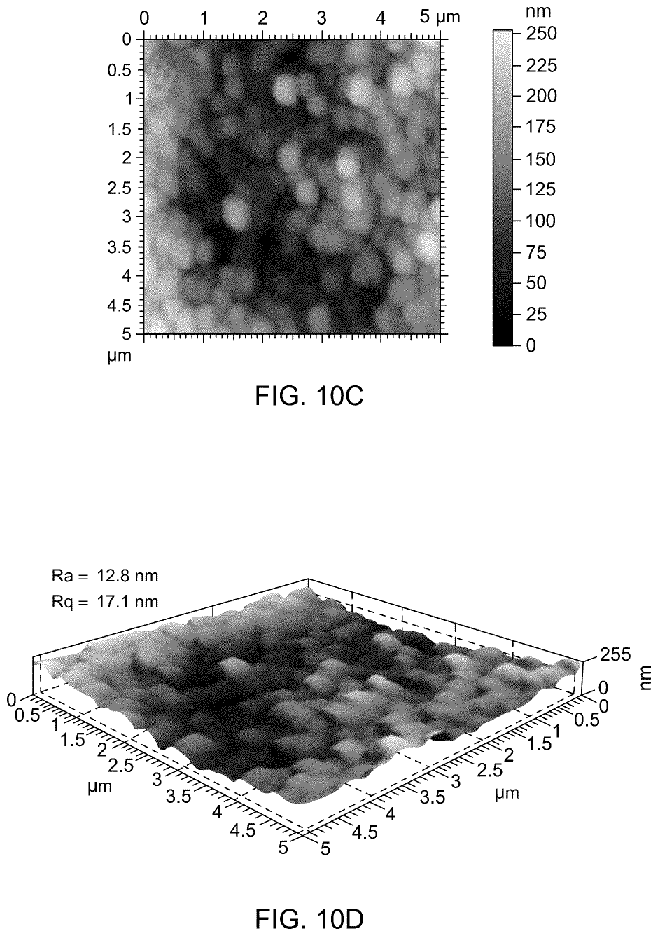

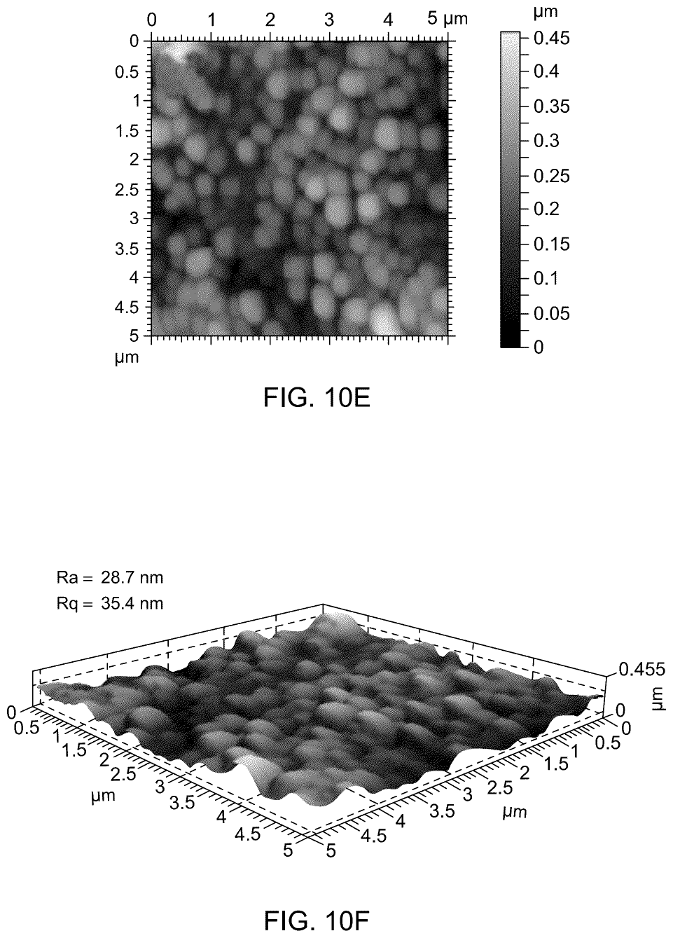

Atomic force microscopy (AFM) is used to measure the surface roughness of the PAN support (FIGS. 10A-10B), 4A-TPC@crosslinked PAN membrane (FIGS. 10C-10D), and the 4A-3P-TPC@crosslinked PAN membrane (FIGS. 10E-10F). As can be seen from FIGS. 10A-10F, the membrane average roughness (Ra) and root mean square roughness (Rq) were found to increase in the following order: crosslinked PAN>4A-TPC@crosslinked PAN membrane>4A-3P-TPC@crosslinked PAN membrane (FIGS. 10A-10F). The Ra value of the 4A-3P-TPC@crosslinked PAN membrane (28.7 nm) was higher than the 4A-TPC@crosslinked PAN membrane (12.8 nm). The higher Ra values in the case of 4A-3P-TPC@ crosslinked PAN membrane were attributed to the presence of larger valleys and deeper ridges than the PAN support and the 4A-TPC-@crosslinked PAN membrane. The presence of ridges and valleys resembles that of commercial polyamide membranes, and this conformation contributes to the rejection of solutes and the permeation of clean permeates through the membranes.

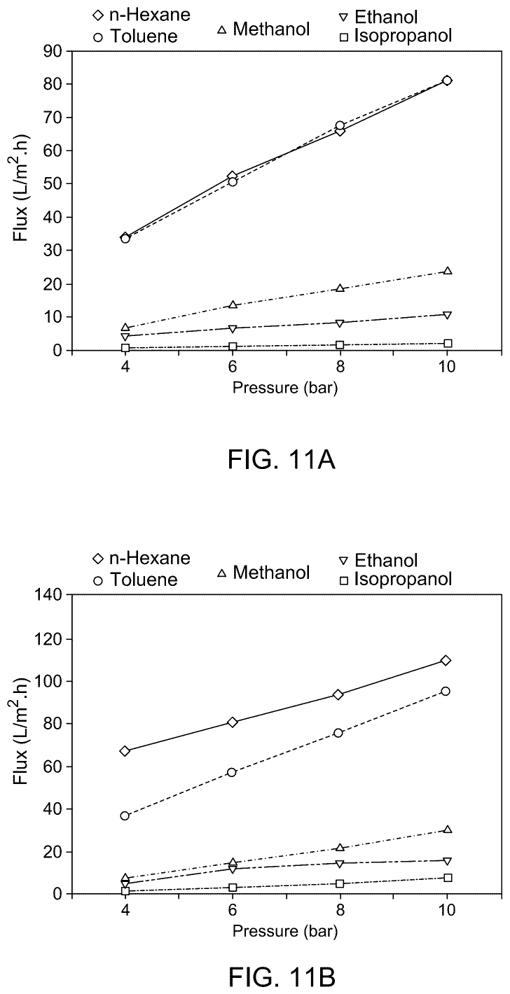

The organic solvent nanofiltration (OSN) performance of the 4A-TPC@crosslinked membrane and the 4A-3P-TPC@crosslinked membrane was studied using a plurality of polar and non-polar solvents as a furtherance in the characterization of membranes. The solvents used may include, but not be limited to, methanol, ethanol, isopropanol, n-hexane, and toluene. The solvents were used as feed in a dead-end filtration cell. Referring to FIG. 11A, a graph depicting the permeate flux versus the transmembrane pressure for the 4A-TPC@crosslinked PAN membrane is illustrated. As can be seen from FIG. 11A, the flux was found to be dependent upon transmembrane pressure, as the permeate flux of the tested solvents increased with increasing transmembrane pressure. The permeate flux of n-hexane and toluene was found to be the highest among the tested solvents. In the case of the 4A-TPC@crosslinked PAN membrane, the permeate flux of n-hexane and toluene was raised from 33.8 liters per meter square hour (L m⁻² h⁻¹) to 81.1 L m⁻² h⁻¹ as the pressure was increased from 4 bar to 10 bar. Referring to FIG. 11B, a graph depicting the permeate flux versus the transmembrane pressure for the 4A-3P-TPC@crosslinked PAN membrane is illustrated. As can be seen from FIG. 11B, the permeate flux of the solvents was found to be higher compared to the 4A-TPC@crosslinked PAN membrane. The n-hexane and toluene showed a flux of 109 L m⁻² h⁻¹ and 95.5 L m⁻² h⁻¹, respectively, at a transmembrane pressure of 10 bar. This might be attributed to the existence of permissible channels for the passage of solvents through the 4A-3P-TPC@crosslinked PAN membrane due to its flexibility for crosslinking during IP. Non-polar organic solvents showed a higher permeate flux than the polar organic solvents. In the case of polar solvents, methanol showed the highest permeate flux.

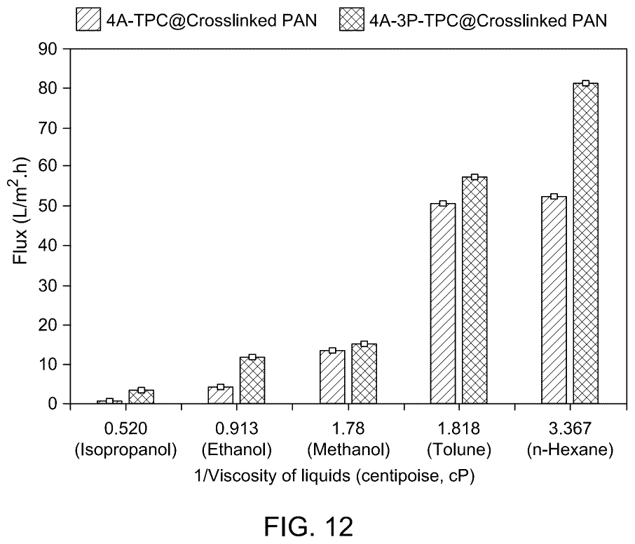

Referring to FIG. 12, a bar graph depicting the effect of the viscosity of solvents on the permeate flux is illustrated. It can be observed from FIG. 12 that as the viscosity of the solvent increases, the permeate flux decreases in an inverse pattern. The n-hexane, with the lowest viscosity of 0.297 centipoise (cP), showed the highest permeate flux, reaching 80 L m⁻² h⁻¹ Isopropanol possessed the lowest permeate flux of 3 L m⁻² h⁻¹ and had the highest viscosity of 1.92 cP.

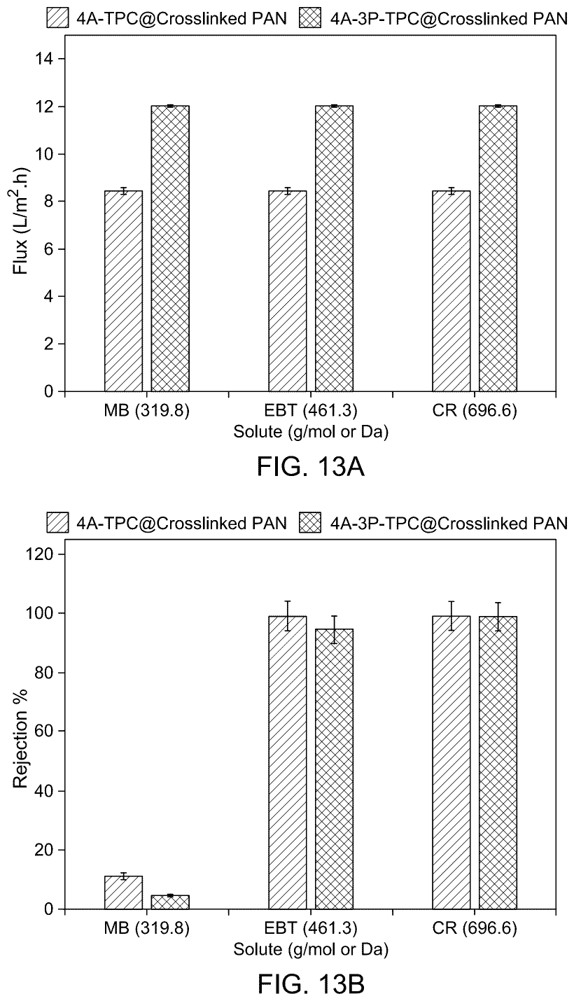

The separation potential of the OSN/SRNF membranes was also studied by using different dyes such as CR, EBT, and MB as model solutes. The solution of each dye was prepared by dissolving an appropriate amount of the dye in methanol at a concentration of 10 ppm. The feed of each dye was loaded in the dead-end cell and filtration was carried out at a pressure of 4 bars. Referring to FIG. 13A, a bar graph depicting the variation of permeate flux as a function of the molecular weight of solutes for membranes is illustrated. During the filtration experiments, the permeate flux of clean methanol was found to be 8.2 L m⁻² h⁻¹ for the 4A-TPC@crosslinked PAN membrane, while a permeate flux of 12 L m⁻² h⁻¹ was seen for the 4A-3P-TPC@crosslinked PAN membrane. Referring to FIG. 13B, a bar graph depicting the rejection performance of the 4A-TPC@crosslinked PAN membrane and 4A-3P-TPC@crosslinked PAN membrane is illustrated. The rejection performance of the membranes was analyzed by recording the absorption spectra of the permeates collected during filtration experiments. The CR showed the highest rejection, reaching 99.1 wt. % for the 4A-TPC@crosslinked PAN membrane and 98.8 wt. % for the 4A-3P-TPC@crosslinked PAN membrane. In the case of EBT, the rejection stayed at 99.1 wt. % for 4A-TPC@crosslinked PAN membrane, while it was slightly reduced for 4A-3P-TPC@Crosslinked PAN membrane, reaching 94.4 wt. %. Although 4A-TPC@crosslinked PAN membrane shows a slightly higher rejection rate than 4A-3_-TPC@crosslinked PAN, the 4A-3P-TPC@crosslinked PAN membrane proved more advantageous compared to the 4A-TPC@crosslinked PAN membrane for removing solutes, as it has a comparatively higher permeate flux with comparable rejection of the solutes. A slight variation in the structure of the reacting monomers during IP alters the performance of the membrane during filtration experiments. The rejection of MB remained considerably lower compared to CR and EBT, with 4.4 wt. % for the 4A-3P-TPC@crosslinked PAN membrane and 11.1 wt. % for the 4A-TPC@crosslinked PAN membrane. The rejection of the dyes was also attempted with non-polar solvents, but the dyes were suspended in the solvent instead of dissolved in a homogeneous solution.

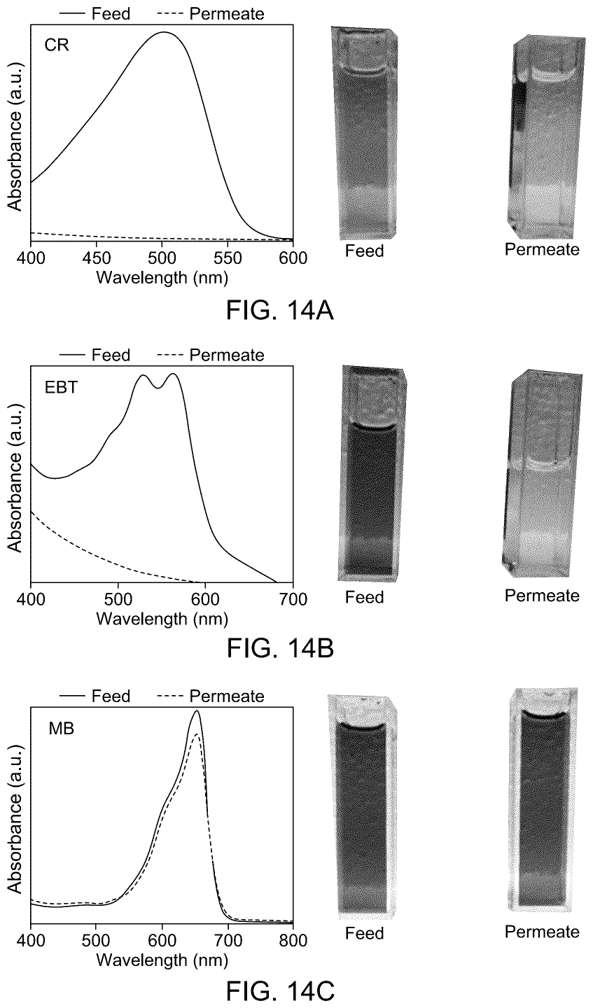

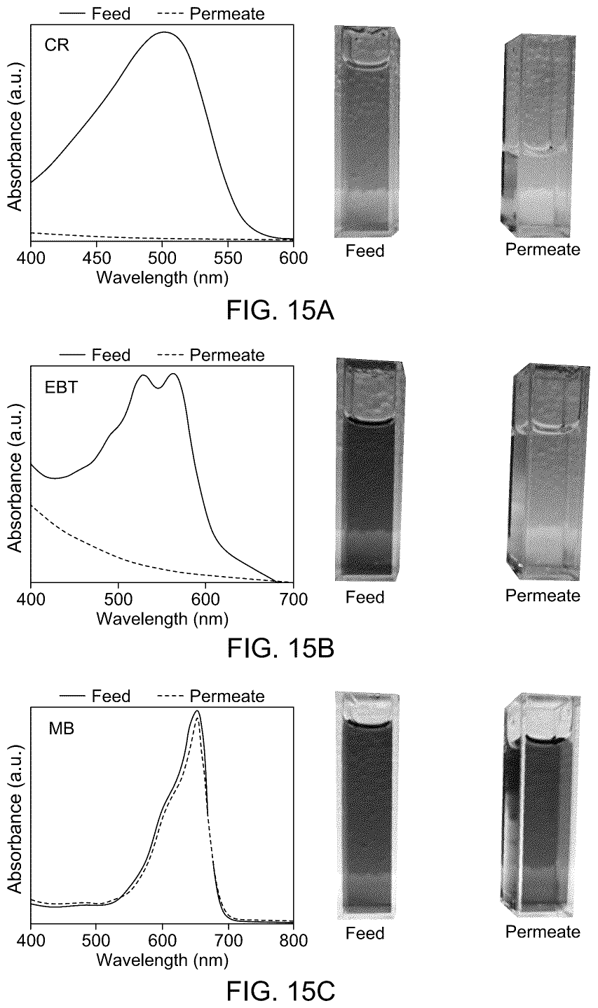

FIGS. 14A-14C depict the comparative results of absorption spectra and coloration of feeds and permeates of CR, EBT, and MB solutes, respectively, for the 4A-TPC@crosslinked membrane, while FIGS. 15A-15C depict the comparative results of absorption spectra and coloration of feeds and permeates of CR, EBT, and MB solutes, respectively, for the 4A-3P-TPC@crosslinked membrane. As can be seen from FIGS. 14A-14C and FIGS. 15A-15C, the absorption maximum of CR at 500 nm was flattened in the case of permeates, which is also reflected by the colorful solution of CR in methanol and the colorless solution of permeates. Similarly, the absorption maximum of EBT at 550 nm was also flattened out in the case of permeates for both membranes. In the case of MB, however, there was not a detectable difference in the absorption spectra and/or coloration of the feed and permeate samples. The OSN/SRNF performances of the current membranes have been compared with those of similar membranes from the literature, as given in Table 1. Compared to similar OSN membranes that have been reported in the literature, the 4A-TPC@crosslinked PAN and 4A-3P-TPC@crosslinked PAN membranes showed higher performance in terms of permeate flux of pure solvents and rejection of solutes, especially CR and EBT; however, the rejection of MB was found to be less compared to the membranes reported in the literature This might be due to the specific molecular weight cut-off (MWCO) of the 4A-TPC@crosslinked PAN and 4A-3P-TPC@crosslinked PAN membranes, as MB has a lower molecular weight than CR and EBT.

TABLE 1
A comparison of PA(TEPA-TCL) @PSU/PETP
membrane with the OSN/SRNF membranes.

| | | | Solute and | |
|---|---|---|---|---|
| | | | molecular | |
| | | Flux | weight | Rejection |
| Membrane | Solvent | (Lm⁻²h⁻¹) | (g mol⁻¹) | (%) |
| Molecularly porous | Methanol | 4.5 | CR (696.55) | >94 |
| hyper-cross-linked | | | EBT (461.81) | |
| polyamide TFC-NF | | | MB (319.85) | >72 |
| membrane^{a} | | | | >90 |
| PA@PS/PET | Methanol | 3.69 | CR (696.55) | >95 |
| TFC-NF | | | EBT (461.81) | >96 |
| membrane^{b} | | | MB (319.85) | >67 |
| PBI^{c} | Ethanol | ~3.0 | CR (696.55) | >60 |
| PBX^{c} | Ethanol | ~2.0 | CR (696.55) | >75 |
| PCX^{c} | Ethanol | ~2.5 | CR (696.55) | >70 |
| PMC^{c} | Ethanol | ~3.2 | CR (696.55) | >70 |
| PBC^{c} | Ethanol | ~1.8 | CR (696.55) | >65 |
| PCS^{c} | Ethanol | ~1.9 | CR (696.55) | >70 |
| PMS^{c} | Ethanol | ~2.0 | CR (696.55) | >70 |
| PA(TEPA- | Methanol | 6 | CR (696.55) | 99.91 |
| TCL)@PSU/PETP | | | EBT (461.81) | 96.92 |
| membrane^{d} | | | MB (319.85) | 87.85 |
| 4A- | Methanol | 8.2 | CR (696.55) | 99.1 |
| TPC@crosslinked | | | EBT (461.81) | 99.1 |
| PAN membrane^{e} | | | MB (319.85) | 11.1 |
| 4A-3P- | Methanol | 12 | CR (696.55) | 98.8 |
| TPC@crosslinked | | | EBT (461.81) | 94.4 |
| PAN membrane^{e} | | | MB (319.85) | 4.40 |

^{a}refers to Waheed, A., Baig, U., and Aljundi, I. H. Fabrication of molecularly porous hyper-cross-linked thin film composite nanofiltration membrane using cyclic amine and linear cross-linker for highly selective organic solvent nanofiltration. Colloid Interface Sci. Commun. 45, 100530, 2021;
^{b}refers to Waheed, A., Abduljawad, S., and Baig, S. Design and fabrication of polyamine nanofiltration membrane by constituting multifunctional aliphatic linear and trifunctional cyanuric chloride for selective organic solvent nanofiltration. J. Taiwan Inst. Chem. Eng. 131, 104204, 2022;
^{c}refers to Beshahwored, S. S., Huang, Y.-H., Abdi, Z. G., Hu, C.-C., and Chung, T.-S. Polybenzimidazole (PBI) membranes cross-linked with various cross-linkers and impregnated with 4-sulfocalix[4]arene (SCA4) for organic solvent nanofiltration (OSN). J. Memb. Sci. 663, 121039, 2022;
^{d}refers to Waheed, A. and Baig, U. Exploiting phase inversion for pentaamine impregnation of ultrafiltration support matrix for rapid fabrication of a hyper-cross-linked polyamide membrane for organic solvent nanofiltration. Process Saf. Environ. Prot. 169, 24-33, 2023; and
^{e}refers to the present disclosure, each of which are incorporated by reference in their entireties.

Successful interfacial polymerization (IP) methods utilized for the fabrication of two thin film organic solvent nanofiltration membranes have been described in the present disclosure. The structure of the polyamide active layers was tuned by slightly altering the chemistry of the reacting amines. Two different linear aliphatic amines, 4A and 4A-3P, were used as aqueous amine solutions during IP on crosslinked PAN support. The resultant organic solvent nanofiltration/solvent resistant nanofiltration membranes, 4A-TPC@crosslinked PAN and 4A-3P-TPC@crosslinked PAN membranes, were thoroughly characterized through scanning electron microscopy, water contact angle, energy disperse X-ray analysis, elemental mapping, attenuated total reflectance Fourier-transform infrared spectrometry, and atomic force microscopy. During organic solvent nanofiltration experiments, the 4A-TPC@crosslinked PAN membrane showed an increase in permeate flux of n-hexane and toluene from 33.8 L m⁻² h⁻¹ to 81.1 L m⁻² h⁻¹ as the pressure was increased from 4 bar to 10 bar. The 4A-3P-TPC@crosslinked PAN membrane possessed n-hexane and toluene flux of 109.9 L m⁻² h⁻¹ and 95.5 L m⁻² h⁻¹ respectively. Congo red showed the highest solute rejection, reaching 99.1 wt. % for the 4A-TPC@crosslinked PAN membrane and 98.8 wt. % for the 4A-3PTPC@crosslinked PAN membrane. In the case of Eriochrome Black T, the rejection stayed at 99.1 wt. % for 4A-TPC@crosslinked PAN membrane, while it was slightly reduced for 4A-3P-TPC@crosslinked PAN membrane, reaching 94.4 wt. %. Based on the rejection and flux data, the ethylene moiety between the two secondary amines in 4A led to a denser active layer compared to the propylene chain between the secondary amine groups of aliphatic amines.

Numerous modifications and variations of the present disclosure are possible in light of the above teachings. It is therefore to be understood that within the scope of the appended claims, the invention may be practiced otherwise than as specifically described herein.

1: A filtration membrane, comprising, in the following order:
a thermoplastic substrate,
a first layer comprising a polyacrylonitrile grafted with a diamine,
a second layer comprising reacted units of a tetramine and reacted units of a phthaloyl chloride,
wherein the tetramine has two secondary amine groups and two primary amine groups, wherein the primary amine groups are separated from the secondary amine groups by a C2 or C3 aliphatic group,
wherein the reacted units of the tetramine and the reacted units of phthaloyl chloride form a polyamide,
wherein one or more amine groups of the polyacrylonitrile of the first layer is cross-linked to the second layer through one or more phthaloyl groups of the reacted units of the phthaloyl chloride.

2: The filtration membrane of claim 1, wherein the diamine is a hydrazine.

3: The filtration membrane of claim 1, wherein the reacted units of tetramine and the reacted units of phthaloyl chloride bond through both the primary and secondary amino groups of the tetramine.

4: The filtration membrane of claim 1, wherein the second layer is the form of nanoparticles with a diameter of 20 nm to 300 nm.

5: The filtration membrane of claim 1, wherein the thermoplastic substrate is in the form of interwoven fibers with a diameter of 2 μm to 20 μm.

6: The filtration membrane of claim 1, wherein the thermoplastic substrate has a thickness of 50 μm to 200 μm.

7: The filtration membrane of claim 1, wherein the first layer includes crosslinked polyacrylonitrile in the form of vertical hollow tubes having a diameter of 1 μm to 50 μm and a length of 2 μm to 100 μm.

8: The filtration membrane of claim 1, wherein the second layer has a thickness of 900 nm to 1000 nm.

9: The filtration membrane of claim 1, wherein the tetramine is N,N′bis(2-aminoethyl)-1,3-propanediamine.

10: The filtration membrane of claim 1, wherein the tetramine is N,N′-bis(3-aminopropyl)ethylenediamine.

11: The filtration membrane of claim 1, wherein the membrane has a water contact angle of 55° to 70°.

12: The filtration membrane of claim 1, wherein the membrane has an average roughness of 10 nm to 30 nm.

13: The filtration membrane of claim 1, wherein the membrane has a surface charge of −0.75 mV to −5.50 mV.

14: A method of nanofiltration, comprising:
contacting the nanofiltration membrane of claim 1, with a solution,
wherein the solution comprises one or more polar solvents, one or more non-polar solvents, and one or more solutes,
collecting a permeate passing through the filtration membrane to obtain a purified composition having a reduced amount of the solutes.

15: The filtration membrane of claim 1, wherein the membrane has a permeate flux of 1 L m⁻² h⁻¹ to 80 L m⁻² h⁻¹ at a pressure of 4 bar.

16: The method of claim 14, wherein the polar solvent is methanol, and the membrane has a permeate flux of 7 L m⁻² h⁻¹ to 13 L m⁻² h⁻¹ at a pressure of 4 bar.

17: The method of claim 14, wherein the polar solvent is methanol, and the membrane has a rejection percentage of the pollutants of 4% to 100% based on an initial weight of the pollutants.

18: The method of claim 14, wherein the pollutants are selected from a group consisting of methylene blue (MB), eriochrome black T (EBT), and Congo red (CR).

19: The method of claim 14, wherein the pollutant is EBT, and the membrane has a rejection percentage of the EBT of 93% to 100% based on an initial weight of the EBT.

20: The method of claim 14, wherein the pollutant is CR, and the membrane has a rejection percentage of the CR of 98% to 100% based on an initial weight of the CR.
